SEQUENCE LISTING (6 sequences)

SEQ ID NO: 1 - protein, 12 aa, Artificial Sequence
  Synthetic Construct
TSFAEYWALLSP

SEQ ID NO: 2 - protein, 12 aa, Artificial Sequence
  Synthetic Construct
VGFAEAWKAQFP

SEQ ID NO: 3 - protein, 78 aa, Artificial Sequence
  Synthetic Construct
MVDPTSFAEYWALLSPDSEPPRMELRSVGDIEQELERAKASIRRLEQEVNQERFRMIYLQTLLAKEKKSYDRRRRRRR

SEQ ID NO: 4 - protein, 78 aa, Artificial Sequence
  Synthetic Construct
MVDPVGFAEAWKAQFPDSEPPRMELRSVGDIEQELERAKASIRRLEQEVNQERFRMIYLQTLLAKEKKSYDRRRRRRR

SEQ ID NO: 5 - protein, 72 aa, Artificial Sequence
  Synthetic Construct
MVDPTSFAEYWALLSPDSEPPRMELRSVGDIEQELERAKASIRRLEQEVNQERFRMIYLQTLLAKEKKSYDR

SEQ ID NO: 6 - protein, 72 aa, Homo sapiens
MVDPVGFAEAWKAQFPDSEPPRMELRSVGDIEQELERAKASIRRLEQEVNQERFRMIYLQTLLAKEKKSYDR

(19) United States
(12) Patent Application Publication
(10) Pub. No.: US 2022/0127335 A1
(43) Pub. Date: Apr. 28, 2022

(54) TETRAMERIC PROTEIN SCAFFOLDS AS NANO-CARRIERS OF THERAPEUTIC PEPTIDES FOR TREATING CANCER AND OTHER DISEASES

(71) Applicant: UNIVERSITY OF MARYLAND, BALTIMORE, Baltimore, MD (US)

(72) Inventors: Wuyuan LU, Baltimore, MD (US); Bohan MA, Yanta District, Xi'an (CN)

(21) Appl. No.: 17/432,568

(22) PCT Filed: Mar. 6, 2020
(86) PCT No.: PCT/US20/21407

§ 371 Date: Aug. 20, 2021

Related U.S. Application Data
(60) Provisional application No. 62/814,574, filed on Mar. 6, 2019.

(51) Int. Cl. C07K 14/82; A61P 35/00; C07K 14/47

(52) U.S. Cl. CPC C07K 14/82; A61P 35/00; C07K 2319/03; C07K 14/4747; C07K 14/4746

(57) ABSTRACT

A protein-based peptide drug carrier derived from the tetramerization domain of the chimeric oncogenic protein Bcr/Abl of chronic myeloid leukemia. Peptides to be delivered are grafted to the N-terminal helical region of Bcr/Abl tetramer. To facilitate cellular uptake, an Arg-repeating hexapeptide is added to the C-terminal end of the Bcr/Abl protein. The protein-based delivery strategy provides a clinically viable solution to p53-inspired anticancer therapy and is applicable to the development of many other peptide therapeutics to target other intracellular protein-protein interactions responsible for disease initiation and progression.

### CROSS-REFERENCE TO RELATED APPLICATIONS

This application claims priority to U.S. Provisional Patent Application No. 62/814,574, filed 6 Mar. 2019 and entitled “Tetrameric Protein Scaffolds as Nano-Carriers of Therapeutic Peptides for Treating Cancer and Other Diseases” in the name of Wuyuan L U et al., incorporated herein by reference in its entirety.

### STATEMENT OF FEDERALLY SPONSORED RESEARCH

This invention was made with government support under CA219150 and CA167296 awarded by The National Institutes of Health. The government has certain rights in the invention.

### BACKGROUND

Intracellular protein-protein interactions (PPIs) control many essential cellular pathways implicated in human diseases [1, 2], representing an important class of therapeutic targets that are considered to be The Holy Grail in drug discovery and development [3, 4]. Among various PPI inhibitors with therapeutic potential, small peptides, compared with low molecular weight compounds, often excel due to their high potency and selectivity and low toxicity [5, 6]. However, major pharmacological disadvantages of peptide inhibitors exist. For example, peptides are susceptible to enzymatic degradation because they generally do not possess a stable tertiary structure to confer resistance to proteolysis; peptides also lack the ability to actively traverse the cell membrane, thus failing to reach intracellular drug targets. Poor proteolytic stability and membrane permeability severely limit peptide bioavailability and therapeutic efficacy [5, 7]. Various elaborate medicinal chemistry approaches and peptide delivery techniques have been developed to overcome these pharmacological barriers [8-16]. While considerable success has been achieved in using peptides to target intracellular PPIs [17-21], much still remains to be done to fulfill their full therapeutic potential.

Nanotechnology has been widely used in the development of new strategies for drug delivery and cancer therapy [22, 23]. Nanoparticle-based traditional delivery tools include, but are not limited to, micelle, liposome, dendrimer, gold nanoshell, and polymer [24, 25]. As unique biopolymers in the nanoscale, proteins are superior in many aspects as a drug carrier to synthetic polymers [26, 27]. Protein-based drug carriers are attractive also because they are amenable to both biological and chemical modifications so that their properties such as molecular size, site of conjugation, and loading capacity can be controlled [28]. In addition, novel functionalities can be engineered into proteins to facilitate cellular uptake and improve targeting specificity. Albumin, a natural transport protein with multiple ligand binding sites, cellular receptor engagement, and a long circulatory half-life, represents a clinically proven platform for the delivery of various drug molecules [29, 30]. Despite the obvious advantages of protein-based drug delivery of low molecular weight compounds, it remains challenging to efficiently deliver peptide therapeutics to target intracellular PPIs.

Accordingly, new approaches are needed for intracellular delivery of peptide therapeutics, for example, to therapeutically disrupt intracellular protein-protein interactions involved in cancer and other diseases. The present description relates to the use of molecular grafting approaches to design a stable protein scaffold with multiple functionalities for the intracellular delivery of peptide therapeutics.

### SUMMARY

The present invention relates to a stable protein scaffold with multiple functionalities for delivering peptide therapeutics to the interiors of cells.

In one aspect, the invention relates to a protein comprising a protein scaffold and at least one therapeutic peptide grafted therein, wherein the protein scaffold is a disulfide-devoid tetramerization domain of chimeric oncoprotein Bcr/Abl protein of chronic myeloid leukemia, as defined in SEQ ID NO: 6.

In another aspect, the invention relates to a ^{PMI}Bcr/Abl protein comprising a sequence as shown in SEQ ID NO: 5.

In still another aspect, the invention relates to a ^{PMI}Bcr/Abl-R6 protein comprising a sequence as shown in SEQ ID NO: 3.

In yet another aspect, the invention relates to a method of inhibiting tumor cell growth in a mammal, said method comprising administering a protein to said mammal, wherein said protein comprises a protein scaffold and at least one therapeutic peptide grafted therein, wherein the protein scaffold is a disulfide-devoid tetramerization domain of chimeric oncoprotein Bcr/Abl protein of chronic myeloid leukemia, as defined in SEQ ID NO: 6.

In yet another aspect, the invention relates to a method of inhibiting tumor cell growth in a mammal, said method comprising administering a protein to said mammal, wherein said protein comprises a ^{PMI}Bcr/Abl protein comprising a sequence as shown in SEQ ID NO: 5.

In still another aspect, the invention relates to a method of inhibiting tumor cell growth in a mammal, said method comprising administering a protein to said mammal, wherein said protein is a ^{PMI}Bcr/Abl-R6 protein comprising a sequence as shown in SEQ ID NO: 3.

In yet another aspect, the invention relates to a method of inducing apoptosis of cancer cells in a mammal, said method comprising administering a protein to said mammal, wherein said protein comprises a protein scaffold and at least one therapeutic peptide grafted therein, wherein the protein scaffold is a disulfide-devoid tetramerization domain of chimeric oncoprotein Bcr/Abl protein of chronic myeloid leukemia, as defined in SEQ ID NO: 6.

In yet another aspect, the invention relates to a method of inducing apoptosis of cancer cells in a mammal, said method comprising administering a protein to said mammal, wherein said protein comprises a ^{PMI}Bcr/Abl protein comprising a sequence as shown in SEQ ID NO: 5.

In still another aspect, the invention relates to a method of inducing apoptosis of cancer cells in a mammal, said method comprising administering a protein to said mammal, wherein said protein is a ^{PMI}Bcr/Abl-R6 protein comprising a sequence as shown in SEQ ID NO: 3.

In yet another aspect, the invention relates to a method of treating Philadelphia chromosome-positive acute lymphocytic leukemia (ALL) and/or chronic myelogenous leukemia (CML) in a mammal, said method comprising administering a protein to said mammal, wherein said protein comprises a protein scaffold and at least one therapeutic peptide grafted therein, wherein the protein scaffold is a disulfide-devoid tetramerization domain of chimeric oncoprotein Bcr/Abl protein of chronic myeloid leukemia, as defined in SEQ ID NO: 6.

In yet another aspect, the invention relates to a method of treating Philadelphia chromosome-positive acute lymphocytic leukemia (ALL) and/or chronic myelogenous leukemia (CML) in a mammal, said method comprising administering a protein to said mammal, wherein said protein comprises a ^{PMI}Bcr/Abl protein comprising a sequence as shown in SEQ ID NO: 5.

In still another aspect, the invention relates to a method of treating Philadelphia chromosome-positive acute lymphocytic leukemia (ALL) and/or chronic myelogenous leukemia (CML) in a mammal, said method comprising administering a protein to said mammal, wherein said protein is a ^{PMI}Bcr/Abl-R6 protein comprising a sequence as shown in SEQ ID NO: 3.

In yet another aspect, the invention relates to a method of delivering a p53-activating compound for cancer treatment, said method comprising administering a protein to a mammal, wherein said protein comprises a protein scaffold and at least one therapeutic peptide grafted therein, wherein the protein scaffold is a disulfide-devoid tetramerization domain of chimeric oncoprotein Bcr/Abl protein of chronic myeloid leukemia, as defined in SEQ ID NO: 6.

In yet another aspect, the invention relates to a method of delivering a p53-activating compound for cancer treatment, said method comprising administering a protein to a mammal, wherein said protein comprises a ^{PMI}Bcr/Abl protein comprising a sequence as shown in SEQ ID NO: 5.

In still another aspect, the invention relates to a method of delivering a p53-activating compound for cancer treatment, said method comprising administering a protein to a mammal, wherein said protein is a ^{PMI}Bcr/Abl-R6 protein comprising a sequence as shown in SEQ ID NO: 3.

### DETAILED DESCRIPTION OF THE DRAWINGS

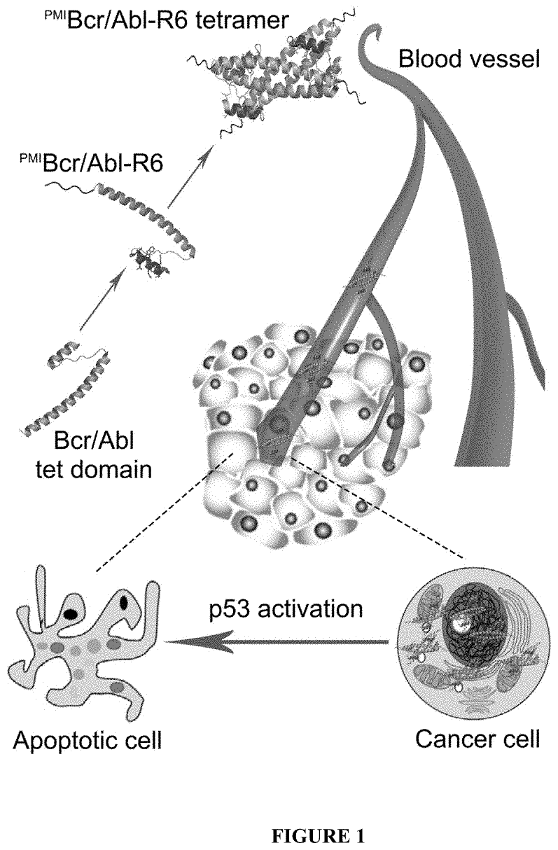

FIG. 1. Strategy for the design of a protein-based nano-carrier of PMI for cancer therapy.

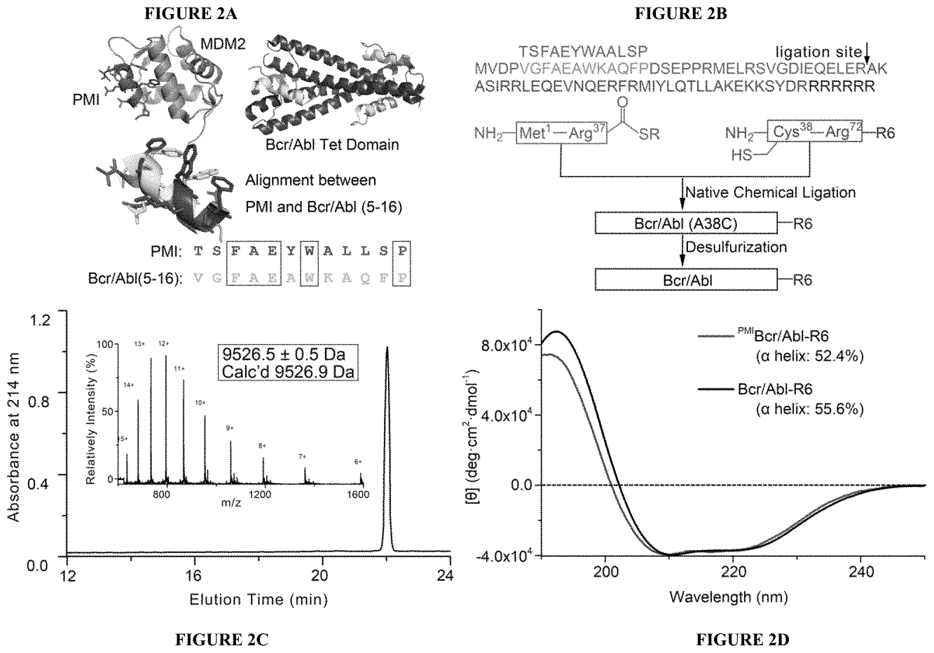

FIG. 2A illustrates the structure-based rational design of ^{PMI}Bcr/Abl-R6. The crystal structures of PMI (red) in complex with MDM2 (green) [39] and of the tetramerization domain of Bcr/Abl (blue/yellow) [34] are shown in ribbons.

FIG. 2B illustrates the total chemical synthesis of ^{PMI}Bcr/Abl, Bcr/Abl-R6 and ^{PMI}Bcr/Abl-R6 via native chemical ligation [40, 41]. All peptides were synthesized on appropriate resin using Boc-chemistry solid phase peptide synthesis [66]. Ligation reactions were carried out in 0.1 M phosphate buffer containing 6 M GuHCl, 100 mM MPAA and 40 mM TCEP, pH 7.4. Desulfurization of the ligation product was achieved by dissolving the peptide at 1 mg/mL in 0.1 M phosphate buffer containing 6 M GuHCl, 0.01 M VA-044, 0.5 M TCEP, 20% t-BuSH.

FIG. 2C illustrates ^{PMI}Bcr/Abl-R6 analyzed by HPLC and electrospray ionization mass spectrometry (ESI-MS). Analytical HPLC was performed on a reversed-phase C18 column (Waters XBridge™ 3.5 μm, 4.6×150 mm) at 40° C.

FIG. 2D illustrates circular dichroism (CD) spectra of Bcr/Abl-R6 (black) and ^{PMI}Bcr/Abl-R6 (red) at 20 μM in 20 mM phosphate buffer, pH 7.4, obtained on a Jasco spectrometer at 25° C. Proteins were quantified spectroscopically by UV measurements at 280 nm using a molar extinction coefficient of 9970 calculated as described [67]. Percent helicity was calculated from the ratio of [θ]₂₂₂ to [θ]ₘₐₓ, where [θ]ₘₐₓ=−39500×[1−(2.57/n)] [68].

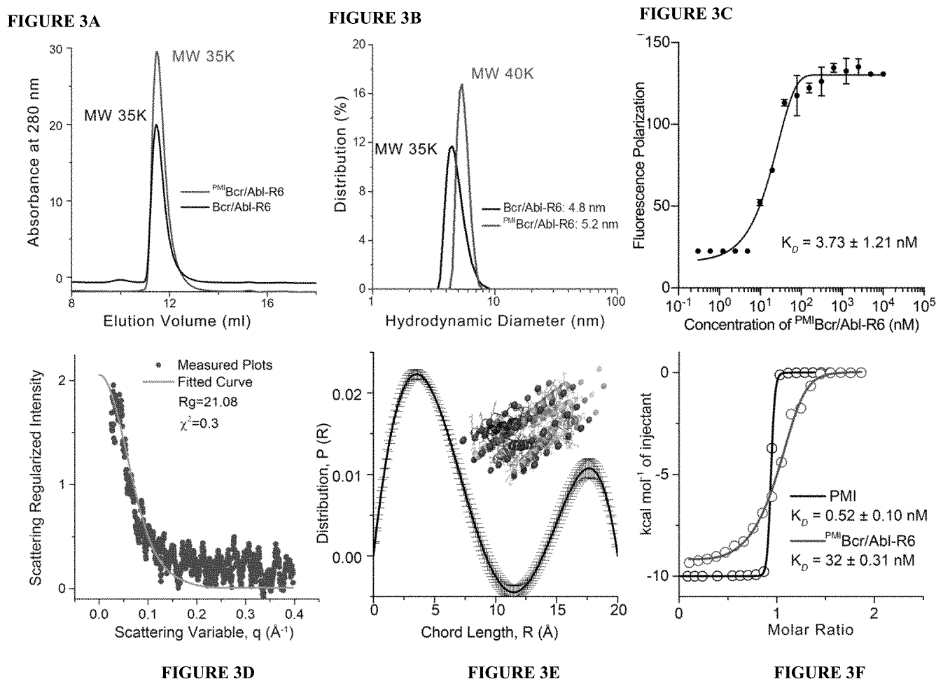

FIG. 3A illustrates size exclusion chromatography of Bcr/Abl-R6 (black) and ^{PMI}Bcr/Abl-R6 (red) performed on a GE Superdex 75 column (10/300 GL) running PBS at a flow rate of 0.5 ml/min at room temperature. The apparent molecular weights of Bcr/Abl-R6 and ^{PMI}Bcr/ABL-R6 were calculated according to a standard calibration curve (not shown), indicating that they exist in aqueous buffer as tetramers.

FIG. 3B illustrates the dynamic light scattering analysis of Bcr/Abl-R6 (black) and ^{PMI}Bcr/Abl-R6 (red) at 20 μM in PBS performed on a Malvin Zetasizer Nano system. The apparent molecular weights were calculated using manufacturer-supplied software.

FIG. 3C illustrates the monomer-tetramer equilibrium of serially diluted ^{PMI}Bcr/Abl-R6 (from 10 μM to 0.3 nM in 20 mM Tris/HCl, pH 7.4) measured in 386-well black plates by fluorescence polarization, yielding a K_{D} value of 3.73±1.21 nM (K_{D}=(monomer)⁴/(tetramer), where the concentrations of monomeric and tetrameric ^{PMI}Bcr/Abl-R6 were derived from fluorescence polarization values). ^{PMI}Bcr/Abl-R6 was N-terminally labeled with a fluorophore, BDP TR (Excitation 589 nm, Emission 616 nm).

FIG. 3D illustrates small angle X-ray scattering (SAXS) diffractograms of ^{PMI}Bcr/Abl-R6 measured at 20 μM in PBS. The orange line is the least squares fit to the data (green points) using a rod model.

FIG. 3E illustrates SAXS analysis of ^{PMI}Bcr/Abl-R6 at 10 μM in PBS at room temperature. The chord length distribution that describes the size, shape and spatial arrangement of ^{PMI}Bcr/Abl-R6 was obtained from SAXS data. The simulated structure of ^{PMI}Bcr/Abl-R6 is largely in agreement with the crystal structure of tetrameric Bcr/Abl (PDB code: 1K1F [34]) (inset).

FIG. 3F illustrates the measurements of the binding affinity of PMI and ^{PMI}Bcr/Abl-R6 for MDM2 in 20 mM Tris/HCl, pH 7.4, by isothermal titration calorimetry on a MicroCal ITC 200 instrument at 25° C. Titrations were carried out by 20 stepwise injections, 2 μL at a time, of 80 μM ^{PMI}Bcr/Abl-R6 in the syringe to 8 μM MDM2 in the cell. For the PMI-MDM2 interaction, the concentrations were 100 μM and 10 μM, respectively. Data were analyzed using the MicroCal Origin program. The K_{D} value of 0.52 nM, measured as described [39], is nearly identical to the published value of PMI determined by surface plasmon resonance [38].

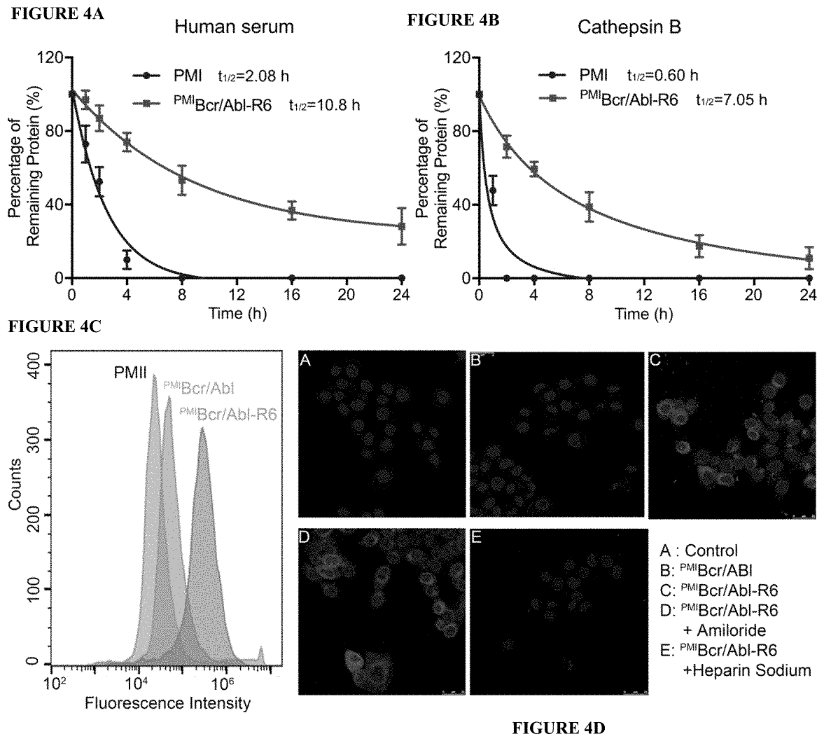

FIG. 4A shows the degradation kinetics of PMI and ^{PMI}Bcr/Abl-R6 at 1 mg/ml in 20 mM Tris-HCl, pH 7.4, containing 10% human serum from a healthy donor. Intact peptide and protein were verified by ESI-MS and quantified by analytical C18 HPLC.

FIG. 4B shows the degradation kinetics of PMI and ^{PMI}Bcr/Abl-R6 at 1 mg/ml in 20 mM sodium acetate buffer, pH 5.0, containing cathepsin B at 10 units/ml.

FIG. 4C illustrates the cellular uptake of PMI, ^{PMI}Bcr/Abl and ^{PMI}Bcr/Abl-R6 analyzed by flow cytometry. PMI, ^{PMI}Bcr/Abl and ^{PMI}Bcr/Abl-R6 were N-terminally labeled with BDP TR (excitation 589 nm, emission 616 nm). HCT 116 p53^{+/+} cells were seeded in a 12-well plate at a density of 30,000 cells/well, cultured for 24 h, and treated with peptide or protein at 10 μM for 4 h before flow cytometric analysis.

FIG. 4D shows the cellular uptake of BDP TR-labeled PMI, ^{PMI}Bcr/Abl and ^{PMI}Bcr/Abl-R6 by HCT 116 p53^{+/+} cells, treated by peptide or protein at 10 μM each for 4 h, and visualized by a confocal laser scanning microscope (panels A-C). Hoechst 33342 blue dye was used for nuclei staining. For the experiments presented in panels D-E, amiloride (3 mM) or heparin sodium (5 mM) was incubated with cells for 12 h before the addition of ^{PMI}Bcr/Abl-R6.

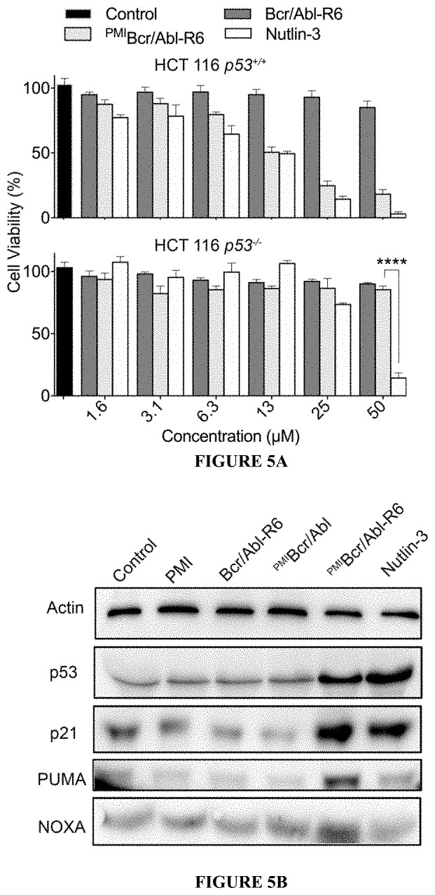

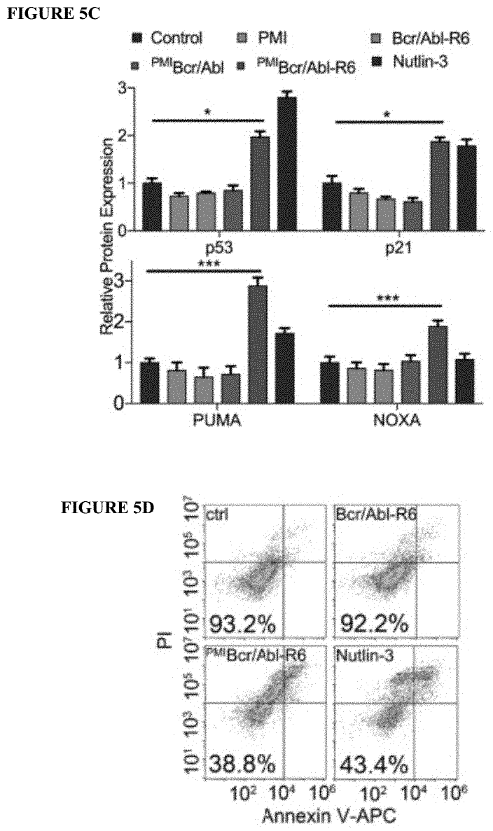

FIG. 5A illustrates the cell viability of HCT116 p53^{+/+} and HCT116 p53^{-/-} cells (3×10³ cells/well in McCoys's 5A medium with 10% FBS) 48 h after treatment with varying concentrations of Bcr/Abl-R6, ^{PMI}Bcr/Abl-R6 and Nutlin-3. Following a 2-h incubation with CCK-8 reagents, absorbance values at 450 nm were measured on a microplate reader, and percent cell viability was calculated as (Aₜᵣₑₐₜₘₑₙₜ−A_{blank})/(A_{control}−A_{blank})×100%. The data are the means of three independent assays. Except for HCT116 p53^{-/-} cells treated by ^{PMI}Bcr/Abl-R6 and Nutlin-3 at 50 μM (****, p<0.0001), no statistically significant difference in activity between ^{PMI}Bcr/Abl-R6 and Nutlin-3 was found.

FIG. 5B shows the representative Western blotting analysis of p53, p21, PUMA, NOXA in HCT116p53^{+/+} cells (2×10⁴ cells/well) 48 h after treatment with PMI, ^{PMI}Bcr/Abl, ^{PMI}Bcr/Abl-R6, Bcr/Abl-R6 and Nutlin-3 at 12.5 μM each, normalized to β-actin. The primary antibodies were from Santa Cruz Biotechnology (p53), Calbiochem (p21, PUMA and NOXA) and Sigma-Aldrich (β-actin), and secondary antibodies conjugated with horseradish peroxidase from Calbiochem.

FIG. 5C shows the quantitative Western blotting analysis (via Image J software) of HCT116 p53^{+/+} cells treated with PMI, Bcr/Abl-R6, ^{PMI}Bcr/Abl, ^{PMI}Bcr/Abl-R6 and Nutlin-3 at 12.5 μM for 48 h. T-test was performed for statistical analysis, * standing for p<0.05, *** for p<0.001. The data are the means±SD of three independent Western blotting assays.

FIG. 5D shows the representative data on apoptosis of HCT116 p53^{+/+} cells 48 h after treatment with Bcr/Abl-R6, ^{PMI}Bcr/Abl-R6 and Nutlin-3 as analyzed by flow cytometry. Cells were seeded in a 12-well plate with a density of 20,000/well and treated with 12.5 μM ^{PMI}Bcr/Abl-R6, Bcr/Abl-R6 or 10 μM Nutlin-3. Apoptosis was detected using a standard apoptotic kit from Biolegend, including APC labeled anti-annexin V antibody and a propidium iodide solution.

FIG. 5E shows the statistical analysis of apoptosis of HCT116 p53^{+/+} cells quantified by flow cytometry. Three independent FACS assays were performed, and data are shown as the means±SD (n=3). p values were calculated by t-test (***, p<0.001).

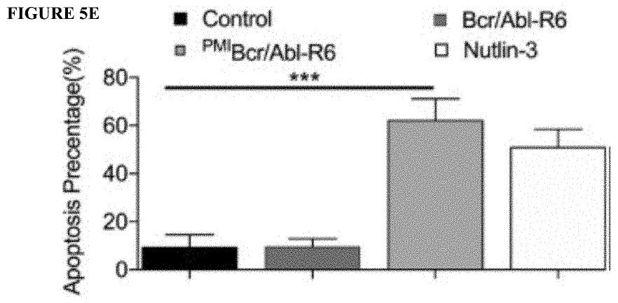

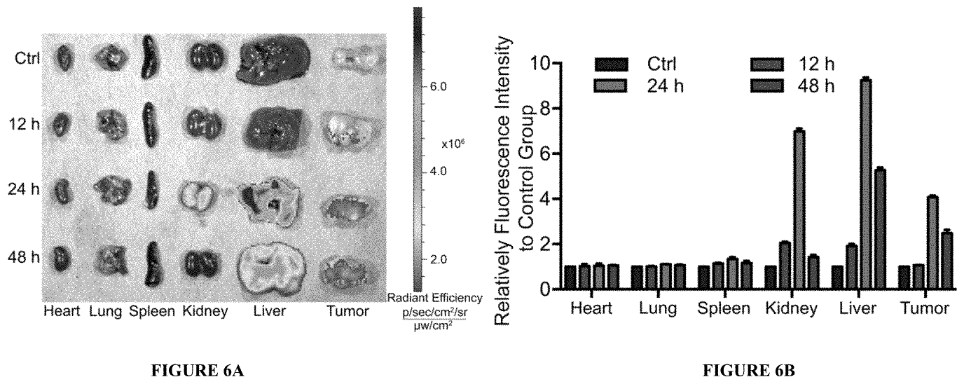

FIG. 6A shows the representative ex vivo fluorescence images of major organs and tumors 12 h, 24 h, 48 h after subcutaneous injection of BDP TR-labeled ^{PMI}Bcr/Abl-R6. HCT116 p53^{+/+} cells (4×10⁶ cells/site) were injected subcutaneously into BALB/c nude mice of four weeks old. Three weeks after tumor cell inoculation, tumor-bearing mice were each injected with 100 μL BDP TR-labeled ^{PMI}Bcr/Abl-R6 at a dose of 5 mg/Kg, and sacrificed for imaging at indicated time points.

FIG. 6B illustrates the semi-quantitative ex vivo analysis of biodistribution of BDP TR-labeled ^{PMI}Bcr/Abl-R6 in the organs and tumor. Fluorescence intensity in each organ was determined using Living Image 3.0. software from IVIS fluorescence data expressed as radiant efficiency (mean±SD, n=3).

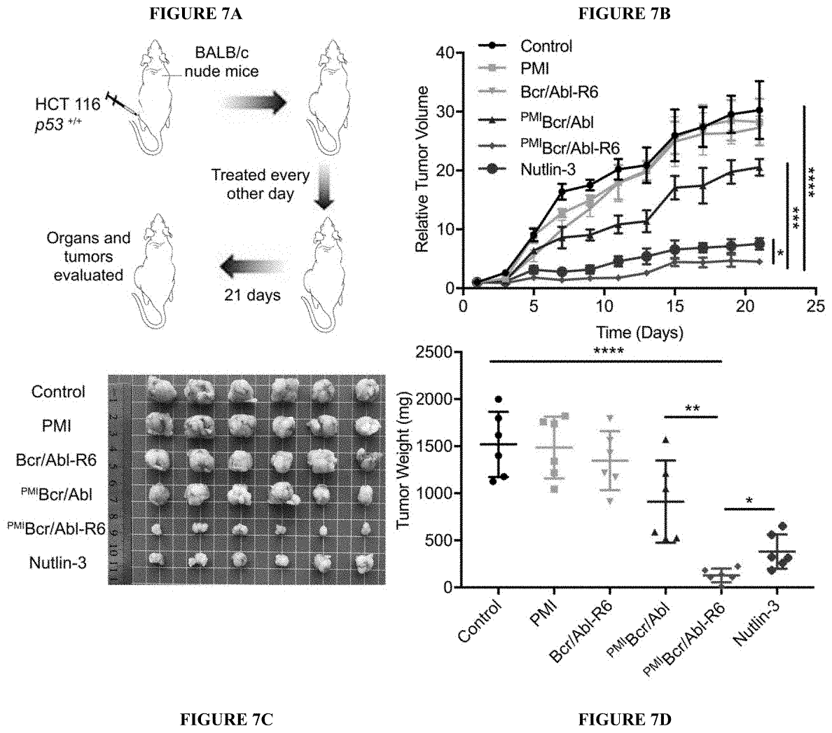

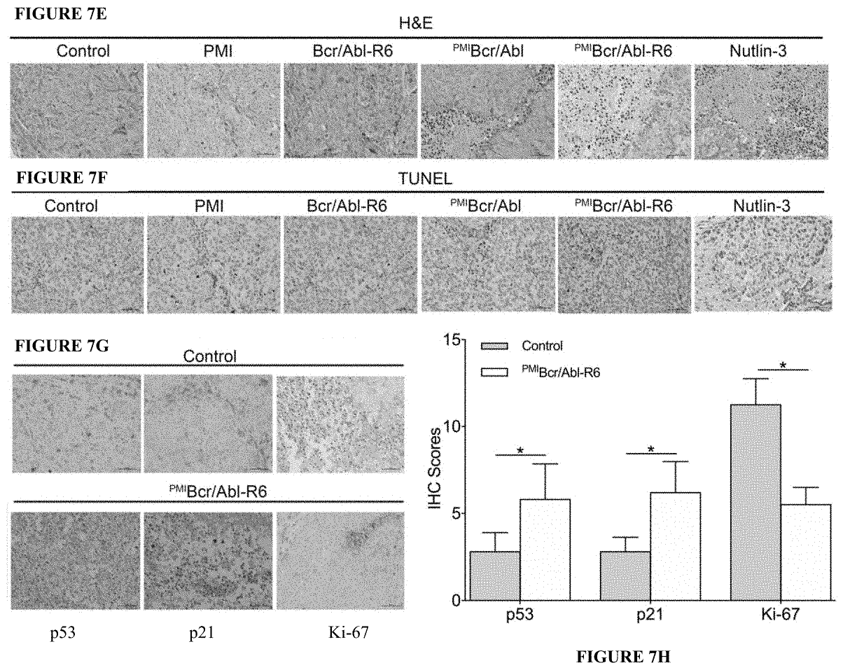

FIG. 7A shows a schematic diagram of therapy. Thirty six athymic nude mice (BALB/c) bearing HCT116 p53^{+/+} xenograft tumors, subcutaneously established in two weeks as a palpable mass (50-100 mm³ in size), were randomly divided into six groups (n=6/group), and treated every other day for three weeks via subcutaneous injection of 20 mM Tris-HCl (mock treatment) and PMI, Bcr/Abl-R6, ^{PMI}Bcr/Abl, ^{PMI}Bcr/Abl-R6 or Nutlin-3 at a dose of 5 mg/Kg.

FIG. 7B illustrates curves of inhibition of tumor growth during the 21-day treatment. Tumor length (L) and width (W) were measured with a caliper, and tumor volume (V) was calculated using the following equation: V=L×W²/2. The data represent the mean±SD (n=6). Statistical analysis was performed using T-test, * standing for p<0.05, *** for p<0.001, and **** for p<0.0001.

FIG. 7C shows images of tumors collected upon conclusion of the three-week treatment.

FIG. 7D illustrates the average weight of tumors excised from each group of mice at the end of treatment. Statistical analysis was performed using T-test, * standing for p<0.05, ** for p<0.01, and **** for p<0.0001.

FIG. 7E shows the histopathological analysis using hematoxylin and eosin (H&E) staining. Representative tumors from each treatment group were fixed with formaldehyde, dehydrated and sliced into 5 μm-thick sections, and subjected to H&E staining according to standard protocols (scale bar: 50 μm).

FIG. 7F is the terminal deoxynucleotidyl transferase dUTP nick end labeling (TUNEL) assay to stain fragmented DNA in apoptotic cells (scale bar: 50 μm).

FIG. 7G shows immunohistochemical (IHC) staining of tumor tissues using commercially available antibodies against p53, p21 and Ki-67 (scale bar: 50 μm). Prepared tissue sections at 5 μm thickness were incubated with various antibodies at 4° C. overnight, and subsequently stained using the Labeled Streptavidin-Biotin (LSAB) staining method. Each stained section was evaluated by a minimum of 10 randomly selected ×20 high-power fields for further statistical analysis.

FIG. 7H is the statistical analysis of IHC scores. A numeric score ranging from 0 to 3 was used to evaluate immunostaining intensity (I): 0, no staining; 1, weak staining; 2, moderate staining; 3, strong staining. To evaluate immunostaining area (A), a numeric score ranging from 1 to 4 was used: 1, positive area<10%; 2, 10%<positive area<50%; 3, 50%<positive area<90%; 4, positive area>90%. The total score, I×A, was calculated accordingly, and statistical analysis was performed using T-test, * standing for p<0.05.

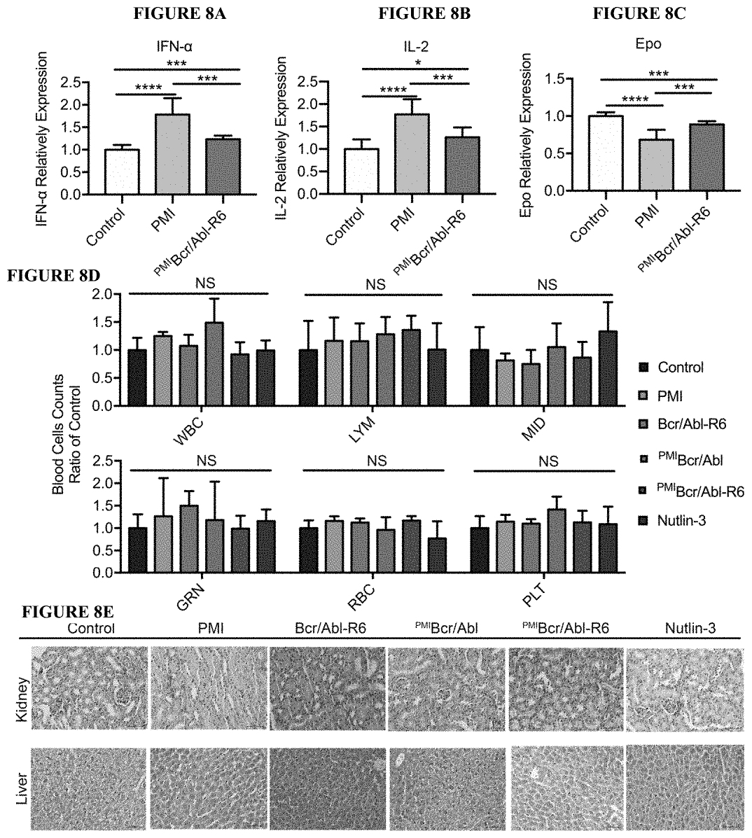

FIG. 8A illustrates the immunogenicity of PMI and ^{PMI}Bcr/Abl-R6 in immune-competent C57BL/6 mice (n=6/group) as measured by the level of IL-2 in the blood in response to subcutaneous treatments with PMI and ^{PMI}Bcr/Abl-R6 for three weeks, every other day, at a dose of 5 mg/Kg. PBS was used as a negative control for mock treatment; IL-2 in the blood collected at the end of the treatment were quantified by ELISA kits (R&D Systems) using protein standards from Sigma-Aldrich. The data from each group are presented as the mean±SD (n=6), and statistical analysis was performed using T-test, * standing for p<0.05, *** for p<0.001, and **** for p<0.0001.

FIG. 8B illustrates the immunogenicity of PMI and ^{PMI}Bcr/Abl-R6 in immune-competent C57BL/6 mice (n=6/group) as measured by the level of TNF-α in the blood in response to subcutaneous treatments with PMI and ^{PMI}Bcr/Abl-R6 for three weeks, every other day, at a dose of 5 mg/Kg. PBS was used as a negative control for mock treatment; TNF-α in the blood collected at the end of the treatment were quantified by ELISA kits (R&D Systems) using protein standards from Sigma-Aldrich. The data from each group are presented as the mean±SD (n=6), and statistical analysis was performed using T-test, * standing for p<0.05, *** for p<0.001, and **** for p<0.0001.

FIG. 8C illustrates the immunogenicity of PMI and ^{PMI}Bcr/Abl-R6 in immune-competent C57BL/6 mice (n=6/group) as measured by the level of erythropoietin in the blood in response to subcutaneous treatments with PMI and ^{PMI}Bcr/Abl-R6 for three weeks, every other day, at a dose of 5 mg/Kg. PBS was used as a negative control for mock treatment; EPO in the blood collected at the end of the treatment were quantified by ELISA kits (R&D Systems) using protein standards from Sigma-Aldrich. The data from each group are presented as the mean±SD (n=6), and statistical analysis was performed using T-test, * standing for p<0.05, *** for p<0.001, and **** for p<0.0001.

FIG. 8D shows the counts of different types of blood cells from a complete blood cell analysis after the 21-day treatment with PMI, Bcr/Abl-R6, ^{PMI}Bcr/Abl, ^{PMI}Bcr/Abl-R6 and Nutlin-3. WBC, white blood cell; LYM, lymphocyte; MID, monocyte; GRN, granulocytes; RBC, red blood cell; PLT, platelet. Statistical analysis was performed using T-test, NS standing for no significant difference.

FIG. 8E illustrates a representative H&E staining of liver and kidney tissues from mice treated with PMI, Bcr/Abl-R6, ^{PMI}Bcr/Abl, ^{PMI}Bcr/Abl-R6 and Nutlin-3 for three weeks (scale bar: 50 μm).

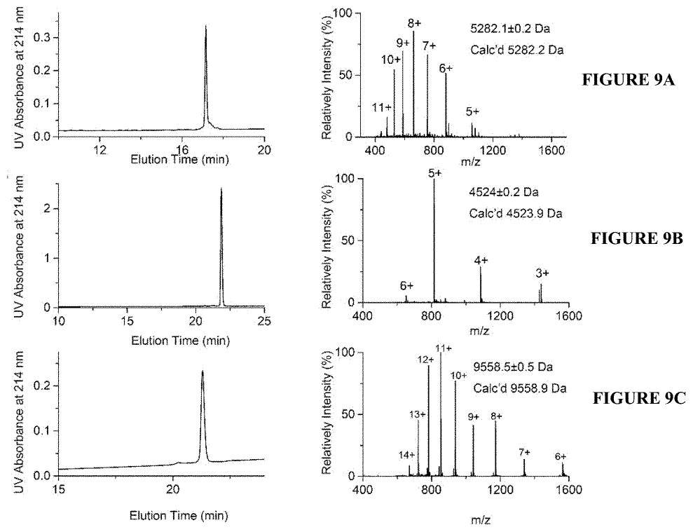

FIG. 9A shows the characterization of C-terminal fragment of ^{PMI}Bcr/Abl-R6 by HPLC and ESI-MS. HPLC analysis was performed at 40° C. on a Waters XBridge C18 reversed phase column (3.5 μm, 4.6×150 mm) running a 30-min gradient of acetonitrile from 5% to 65% at a flow rate of 1 ml/min.

FIG. 9B shows the characterization of N-terminal fragment of ^{PMI}Bcr/Abl-R6 by HPLC and ESI-MS. HPLC analysis was performed at 40° C. on a Waters XBridge C18 reversed phase column (3.5 μm, 4.6×150 mm) running a 30-min gradient of acetonitrile from 5% to 65% at a flow rate of 1 ml/min.

FIG. 9C shows the characterization of the full-length ligation product of ^{PMI}Bcr/Abl-R6 by HPLC and ESI-MS. HPLC analysis was performed at 40° C. on a Waters XBridge C18 reversed phase column (3.5 μm, 4.6×150 mm) running a 30-min gradient of acetonitrile from 5% to 65% at a flow rate of 1 ml/min.

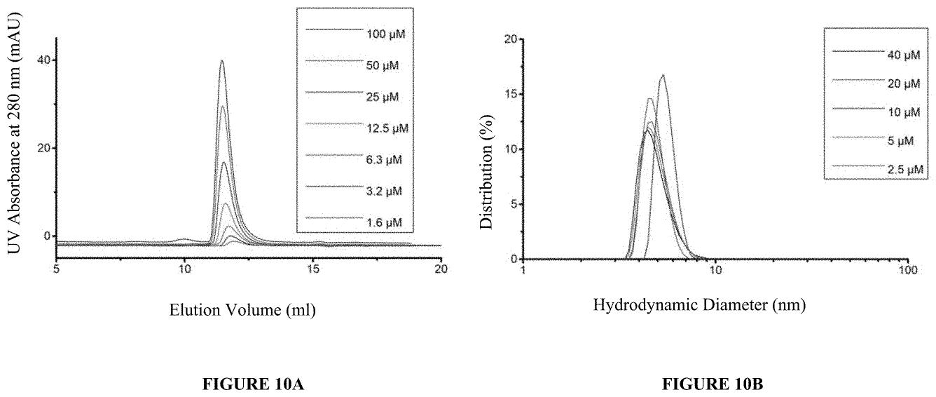

FIG. 10A illustrates the tetramerization of ^{PMI}Bcr/Abl-R6 at different concentrations as determined by size exclusion chromatography.

FIG. 10B illustrates the tetramerization of ^{PMI}Bcr/Abl-R6 at different concentrations as determined by dynamic light scattering.

FIG. 11 shows the zeta potential of ^{PMI}Bcr/Abl-R6 or ^{PMI}Bcr/Abl was measured at 20 μM in PBS on a Zetasizer Nano from Malvern.

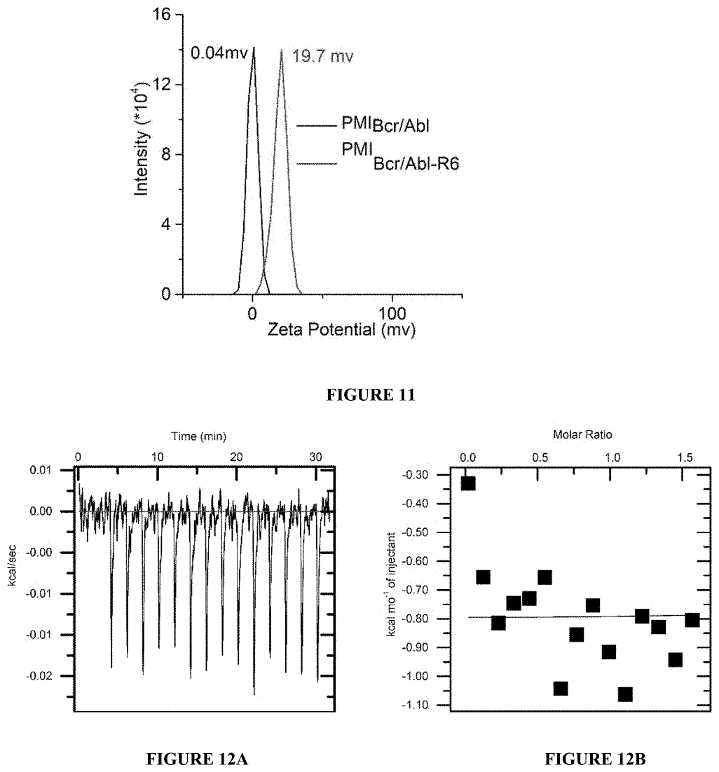

FIG. 12A shows the binding of Bcr/Abl-R6 to MDM2 as measured by ITC. ITC measurements were performed on a MicroCal ITC 200 calorimeter (GE Healthcare) at 25° C. in 20 mM Tris/HCl, pH 7.4. Titrations were carried out by 20 stepwise injections, 2 μL at a time, of 80 μM Bcr/Abl-R6 in the syringe to 8 μM MDM2 in the cell. Data were analyzed using the MicroCal Origin program. No binding was detected between Bcr/Abl-R6 and MDM2.

FIG. 12B shows the binding of Bcr/Abl-R6 to MDM2 as measured by ITC. ITC measurements were performed on a MicroCal ITC 200 calorimeter (GE Healthcare) at 25° C. in 20 mM Tris/HCl, pH 7.4. Titrations were carried out by 20 stepwise injections, 2 μL at a time, of 80 μM Bcr/Abl-R6 in the syringe to 8 μM MDM2 in the cell. Data were analyzed using the MicroCal Origin program. No binding was detected between Bcr/Abl-R6 and MDM2.

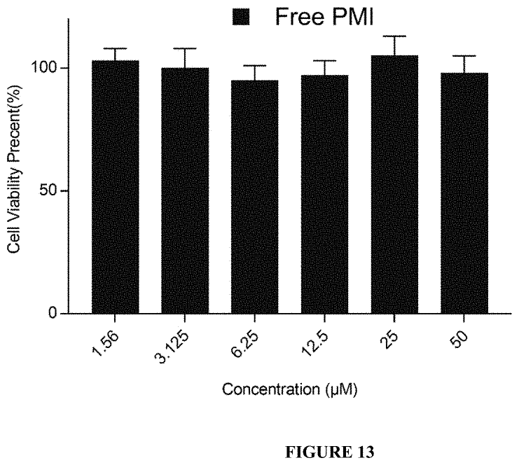

FIG. 13 illustrates the cell viability of HCT116 p53^{+/+} cells 48 h after treatment with free PMI. Three independent assays were performed.

### DESCRIPTION

The present invention relates to a stable protein scaffold with multiple functionalities for delivering peptide therapeutics to the interiors of cells. More specifically, the present invention relates to protein-based peptide drug carrier derived from the tetramerization domain of the chimeric oncogenic protein Bcr/Abl of chronic myeloid leukemia (MVDPVGFAEAWKAQFPDSEPPRMELRSVGDIEQELERAKASIRRLEQEVNQERFRMIYLQTLLAKEK KSYDR; SEQ ID NO: 6).

The p53-MDM2/MDMX interaction has garnered much attention as an important intracellular drug target for the development of MDM2/MDMX antagonists or p53-activating agents for anticancer therapy [37, 55-57]. Small molecule antagonists are generally mono-specific for MDM2, and several are in clinical trials with promising early results [54, 58]. By contrast, peptide antagonists are often dual-specific for both MDM2 and MDMX, potentially affording more robust and sustained p53 activation. One notable example is ALRN-6924, a hydrocarbon-stapled peptide antagonist of MDM2 and MDMX kills tumor cells harboring wild-type p53 in phase 2 clinical trials for advanced solid tumors and lymphomas [59]. More recently, ALRN-6924 has been reported to be effective against acute myeloid leukemia in vitro and in vivo [60]. The hydrocarbon-stapling technique pioneered by Verdine and colleagues enables side-chain cross-linked and conformationally stabilized helical peptides to traverse the cell membrane with improved proteolytic stability and enhanced biological activity [61, 62]. Of note, a hydrocarbon- or dithiocarbamate-stapled PMI (p53-MDM2/MDMX inhibitor) has been shown to be a potent p53 activator in vitro and in vivo [63-65]. Despite these successes, it is worth noting that small peptides do not have a sufficiently long circulation half-life in vivo due to renal excretion (<20 KDa), thus adversely affecting their therapeutic efficacy. By contrast, the protein construct ^{PMI}Bcr/Abl-R6 described herein, a stable tetramer of 35 KDa that can be readily prepared in large quantity via recombinant expression, is expected to have excellent bioavailability compared with small peptide therapeutics.

Most protein scaffolds used for peptide grafting are stabilized by disulfide bonds [31-33], and thus are unsuitable for targeting PPIs in the cytoplasmic space where the reducing environment can structurally destabilize disulfide-bridged proteins prompting their proteolytic degradation. To circumvent this severe limitation, the present inventors have identified the disulfide-devoid tetramerization domain of the chimeric oncoprotein Bcr/Abl of chronic myeloid leukemia (CML) [34], which forms a highly stable tetramer in solution, as a protein scaffold for molecular grafting of therapeutic peptides of an α-helical nature.

The present inventors introduced into the N-terminus of Bcr/Abl, a potent dodecameric peptide antagonist, termed PMI, of both MDM2 and MDMX—the two oncogenic proteins that functionally inhibit the tumor suppressor protein p53 in many tumor types [35, 36]. To antagonize intracellular MDM2/MDMX for p53 activation, ^{PMI}Bcr/Abl was extended by a C-terminal Arg-repeating hexapeptide (R6) to facilitate its cellular uptake. The resultant tetrameric protein ^{PMI}Bcr/Abl-R6 adopted an alpha-helical conformation in solution and bound to MDM2 at an affinity of 32 nM. ^{PMI}Bcr/Abl-R6 effectively induced apoptosis of HCT116 p53^{+/+} cells in vitro in a p53-dependent manner and potently inhibited tumor growth in a nude mouse xenograft model by antagonizing MDM2/MDMX to reactivate the p53 pathway. This protein scaffold, Bcr/Abl-R6, can be used as a delivery tool for α-helical peptides to target a great variety of intracellular PPIs for disease intervention. In addition to being generally useful as a protein-based universal carrier for delivering peptide therapeutics for treatment of various diseases, the protein scaffold and methods described herein can be used specifically, e.g., to deliver ^{PMI}Bcr/Abl as a p53-activating compound for cancer therapy, as well as to deliver ^{PMI}Bcr/Abl as a p53-activating and Bcr/Abl-inhibiting compound for the treatment of Philadelphia chromosome-positive acute lymphocytic leukemia (ALL) and/or chronic myelogenous leukemia (CML) resistant to imatinib.

Accordingly, in one aspect, the instant application relates to a protein comprising a protein scaffold and at least one therapeutic peptide grafted therein, wherein the protein scaffold is a disulfide-devoid tetramerization domain of chimeric oncoprotein Ber/Abl protein of chronic myeloid leukemia, as defined in SEQ ID NO: 6. The therapeutic peptide can have an α-helical structure. The therapeutic peptide can be grafted into the N-terminus of the Bcr/Abl protein. The therapeutic peptide can be any p53-activating peptide of an alpha-helical nature and is useful for the treatment of any cancer harboring wild type-p53 and elevated MDM2/MDMX. Further, the therapeutic peptide can be any antitumor peptide of an alpha-helical nature and is useful for treating cancer in general. The therapeutic peptide can be linear or stapled. For example, in one embodiment, the therapeutic peptide is PMI, which antagonizes intracellular MDM2/MDMX, thereby activating p53. The PMI is grafted in place of residues 5-16 of the Bcr/Abl protein. The protein, regardless of the peptide grafted therein, can further comprise a C-terminal extension to allow the protein to traverse a cell membrane. For example, in one embodiment, the C-terminal extension is an Arg-repeating hexapeptide (R6).

In another aspect, the present application relates to a ^{PMI}Bcr/Abl protein comprising a sequence as shown in SEQ ID NO: 5. The ^{PMI}Bcr/Abl protein can further comprise a C-terminal extension to allow the protein to traverse a cell membrane. For example, in one embodiment, the C-terminal extension is an Arg-repeating hexapeptide (R6).

In still another aspect, the present application relates to a ^{PMI}Bcr/Abl-R6 protein comprising a sequence as shown in SEQ ID NO: 3.

In addition to PMI, other therapeutic peptides that can be grafted into the protein scaffold of SEQ ID NO: 6 include, but are not limited to: MTide-01, sMTide-01, MTide-02, sMTide-02, sMTide-02A, sMTide-02B, as disclosed C. J. Brown et al., ACS Chem. Biol., 2013, 8, 506-512 [63], which is incorporated by reference herein in its entirety; PMI(1,5)-a, PMI(1,5)-b, PMI(2,6)-a, PMI(2,6)-b, PMI(4,8)-a, PMI(4,8)-b, PMI(5,9)-a, PMI(5,9)-b, PMI(8,12)-a, and PMI(8,12)-b, as disclosed in Xiang Li et al., Chem. Sci., 2079, 10, 1522, which is incorporated by reference herein in its entirety; and N8A-PMI and other truncated analogs of PMI, as disclosed in Chong Li et al., J. Mol. Biol., 2010, 398(2), 200-213 (doi: 10.1016/j.jmb.2010.03.005).

It should be appreciated that the proteins described herein can be present in a formulation that is suited for administration to the subject. Accordingly, in another aspect, the present application relates to a formulation, said formulation comprising the protein and at least one pharmaceutically acceptable excipient. The formulation can further comprise at least one additional active pharmaceutical ingredient (API) such as an anticancer agent.

The term “pharmaceutically acceptable excipient” refers to a carrier, diluent, or adjuvant which is administered with the proteins described herein. Such pharmaceutically acceptable excipients may be liquid-based, such as water and oils, including those of petroleum, animal, vegetable, or synthetic origin, such as peanut oil, soybean oil, mineral oil, sesame oil, and the like. Water or aqueous salt solutions and aqueous solutions of dextrose and glycerol, particularly for injectable solutions, are preferably used as excipients. Additional pharmaceutically acceptable excipients include, but are not limited to, any and all solvents, buffering agents (such as a phosphate buffer, citrate buffer, and buffers made from other organic acids), dispersion media, surfactants, antioxidants (e.g., ascorbic acid), preservatives (e.g., antibacterial agents, antifungal agents), a polypeptide (such as serum albumin, gelatin, and an immunoglobulin), a hydrophilic polymer (such as polyvinylpyrrolidone), an amino acid (such as glycine, glutamine, asparagine, arginine, and/or lysine), a monosaccharide, a disaccharide, and/or other carbohydrates (including glucose, mannose, and dextrins), a chelating agent (e.g., ethylenediaminetetratacetic acid (EDTA)), a sugar alcohol (such as mannitol and sorbitol), a salt-forming counterion (e.g., sodium), an anionic surfactant (such as TWEEN, PLURONICS, and PEG), isotonic agents, absorption delaying agents, salts, drug stabilizers, gels, lubricants, sweetening agents, flavoring agents, dyes, and combinations thereof, as would be known to one of ordinary skill in the art (see, “Remington Pharmaceutical Sciences” by E W Martin, 21′ Edition, 2005). A pharmaceutically acceptable excipient suitable for use in the formulation and methods described herein is non-toxic to cells, tissues, or subjects at the dosages employed.

The proteins or formulations described herein can be used for ameliorating and/or treating a cancer. In one embodiment, the cancer is related to inactivation and/or mutation of p53. A non-limiting exemplary list of cancers includes, but is not limited to, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia (CLL) including B-CLL, acute myelogenous leukemia, chronic myelogenous leukemia, chronic granulocytic leukemia, acute granulocytic leukemia, hairy cell leukemia, neuroblastoma, sarcoma such as liposarcoma, malignant fibrous histiocytoma, osteosarcoma, Ewing's sarcoma, leiomyosarcoma, and rhabdomyosarcoma, Kaposi's sarcoma, polycythemia vera, essential thrombocytosis, Hodgkin's disease, non-Hodgkin's lymphoma, soft-tissue sarcomas such as lipoma, and malignant Schwannoma, osteogenic sarcoma, primary macroglobulinemia, and retinoblastoma, and the like, T and B cell mediated autoimmune diseases; inflammatory diseases; infections; hyperproliferative diseases; AIDS; degenerative conditions, vascular diseases, and the like. In a preferred embodiment, the cancer ameliorated and/or treated is selected from at least one of melanoma, lung cancer, a sarcoma, colon cancer, prostate cancer, choriocarcinoma, breast cancer, retinoblastoma, stomach carcinoma, acute myeloid leukemia, a lymphoma, multiple myeloma, and a leukemia in a subject. In some embodiments, the cancer cells being treated are metastatic. In other embodiments, the cancer cells being treated are resistant to other anticancer agents.

Treating cancer includes, but is not limited to, reducing the number of cancer cells or the size of a tumor in the subject, reducing progression of a cancer to a more aggressive form, reducing proliferation of cancer cells or reducing the speed of tumor growth, killing of cancer cells, inducing apoptosis of cancer cells, reducing metastasis of cancer cells or reducing the likelihood of recurrence of a cancer in a subject. Treating a subject, as used herein, refers to any type of treatment that imparts a benefit to a subject afflicted with a disease or at risk of developing the disease, including improvement in the condition of the subject (e.g., in one or more symptoms), delay in the progression of the disease, delay the onset of symptoms or slowing the progression of symptoms, etc.

The proteins or formulations and methods related to treating cancer, as described herein, can be used to treat subjects such as mammals (e.g., humans) having cancer. Examples of mammals that can be treated as described herein include, without limitation, humans, monkeys, dogs, cats, cows, horses, pigs, rats, and mice.

The methods of treatment, as described herein, relate to the administration of a therapeutically effective amount of the proteins or formulations, as described herein, to a subject in need of said treatment. As used herein, an “effective amount” or a “therapeutically effective amount” means the amount of a protein that, when administered to a subject for treating cancer is sufficient to effect a treatment (as defined above). The therapeutically effective amount will vary depending on the protein, or formulation comprising same, the disease and its severity and the age, weight, physical condition and responsiveness of the subject to be treated.

It will be appreciated that the specific dosage administered in any given case will be adjusted in accordance with the protein or formulation being administered, the disease to be treated or inhibited, the condition of the subject, and other relevant medical factors that may modify the activity of the protein or formulation or the response of the subject, as is well known by those skilled in the art. For example, the specific dose for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the protein or formulation such as by means of an appropriate conventional pharmacological or prophylactic protocol.

The maximal dosage for a subject is the highest dosage that does not cause undesirable or intolerable side effects. The number of variables in regard to an individual treatment regimen is large, and a considerable range of doses is expected. The route of administration will also impact the dosage requirements. It is anticipated that dosages of the protein scaffold or formulation will reduce growth of the cancer by at least 10%, 20%, 30%, 40%, 50%, 60%, 70%, 80%, 90% or 100% compared to a cancer left untreated.

Suitable effective dosage amounts for administering the protein or formulation may be determined by those of skill in the art, but typically range from about 1 microgram to about 10,000 micrograms per kilogram of body weight weekly, although they are typically about 1,000 micrograms or less per kilogram of body weight weekly. In some embodiments, the effective dosage amount ranges from about 10 to about 10,000 micrograms per kilogram of body weight weekly. In another embodiment, the effective dosage amount ranges from about 50 to about 5,000 micrograms per kilogram of body weight weekly. In another embodiment, the effective dosage amount ranges from about 75 to about 1,000 micrograms per kilogram of body weight weekly. The effective dosage amounts described herein refer to total amounts administered, that is, if more than one protein or formulation is administered, the effective dosage amounts correspond to the total amount administered. The protein or formulation can be administered as a single dose or as divided doses. For example, the protein or formulation may be administered two or more times separated by 4 hours, 6 hours, 8 hours, 12 hours, a day, two days, three days, four days, one week, two weeks, or by three or more weeks.

In various embodiments, the protein or formulation can be administered by intravenous, intraarterial, intrathecal, intradermal, intracavitary, oral, rectal, intramuscular, subcutaneous, intracisternal, intravaginal, intraperitonial, topical, buccal, and/or nasal routes of administration.

Accordingly, in another aspect, the present application relates to a method of inhibiting tumor cell growth in a mammal, said method comprising administering any of the proteins or formulations described herein to said mammal. The protein antagonizes intracellular MDM2/MDMX for p53 activation.

In still another aspect, the present application relates to a method of inducing apoptosis of cancer cells in a mammal, said method comprising administering any of the proteins or formulations described herein to said mammal. The protein antagonizes intracellular MDM2/MDMX for p53 activation.

In still another aspect, the present application relates to a method of treating Philadelphia chromosome-positive acute lymphocytic leukemia (ALL) and/or chronic myelogenous leukemia (CML) in a mammal, said method comprising administering any of the proteins or formulations described herein to said mammal. In one embodiment, the ALL and/or CML are resistant to imatinib.

In another aspect, the present application relates to a method of delivering a p53-activating compound for cancer treatment, said method comprising administering any of the proteins or formulations described herein to said mammal.

In yet another aspect, the invention also relates to the use of a protein or formulation as described herein for the manufacture of a medicament for the treatment of cancer.

In still another aspect, the invention relates to the use of the protein or the formulation as described herein as a medicament.

In yet another aspect, the present invention relates to a method of using recombinant techniques to make any of the proteins as described herein, as readily understood by the person skilled in the art.

It has been demonstrated that the tetrameric Bcr/Abl scaffold is an ideal protein-based nanocarrier of p53-activating peptides to target the p53-MDM2/MDMX interaction for cancer therapy. MDM2 and MDMX cooperate to persistently inhibit p53 function and target the tumor suppressor protein for proteasomal degradation, contributing to tumor development and progression. ^{PMI}Bcr/Abl-R6 as a dual-specificity antagonist of MDM2 and MDMX and a powerful p53 activator in vitro and in vivo is superior in many aspects to mono-specific small molecule inhibitors of MDM2 as well as stapled peptide antagonists currently in clinical trials, promising a novel class of antitumor agents with significant therapeutic potential. Importantly, this protein-based nanocarrier is also suitable for the design of different classes of peptide therapeutics of an α-helical nature to target intracellular PPIs involved in many other human diseases.

It should be appreciated by the person skilled in the art that although reference herein is to the targeting of intracellular proteins, the protein-based nanocarrier is also suitable for the design of different classes of peptide therapeutics of an α-helical nature to target extracellular PPIs involved in many other human diseases as well.

The features and advantages of the invention are more fully shown by the illustrative examples discussed below.

### Example: Tetrameric Protein Scaffold as a Nanocarrier of Therapeutic Peptides for Treating Cancer and Other Diseases

### Design Strategy

In many tumor cells harboring wild type p53, the E3 ubiquitin ligase MDM2 and/or its homolog MDMX (also known as MDM4) block the transcriptional activity of p53 and target the tumor suppressor protein for proteasomal degradation, conferring tumor development and progression [35-37]. MDM2/MDMX antagonism has been validated as an effective therapeutic strategy for cancer treatment. Since MDMX potentiates MDM2 function in p53 inhibition, dual-specificity antagonists of both MDM2 and MDMX are particularly attractive as therapeutic agents for robust and sustained p53 activation [37]. The present inventors previously identified PMI, a series of high-affinity and dual-specificity dodecameric peptide antagonists of MDM2 and MDMX, through combinatorial library screening and structure-based rational design approaches [38, 39]. Although PMI peptides tightly bind, in an α-helical conformation, to the p53-binding pocket of MDM2 and MDMX at affinities ranging from high pM to low nM, they are not inhibitory per se against tumor growth due mainly to their inability to traverse the cell membrane [38, 39].

To carry therapeutic peptides of an α-helical nature for cancer therapy, it has been hypothesized that the protein must meet the following five criteria: (1) structurally amenable to peptide grafting with a pre-existing short α-helix, (2) sufficiently large in size (via oligomerization, for example) to alleviate renal excretion, (3) resistant to proteolytic degradation by adopting a stable structure with few flexible loops and disordered regions, (4) devoid of disulfide bonds, and (5) efficient in membrane permeabilization. Bcr/Abl tetramerization domain comprises 72 amino acid residues and forms a coiled-coil tetramer, with each monomer consisting of a short N-terminal α-helix, a connecting loop, and a long C-terminal α-helix [34]. This protein is thus ideally suited as a nano-carrier of PMI for cancer therapy because it readily meets the first four criteria defined. To enable its membrane permeability, however, additional modifications such as introduction of a cationic penetrating peptide sequence to Bcr/Abl tetramerization domain is warranted. The design strategy is schematically illustrated in FIG. 1.

In FIG. 1, the tetramerization domain of 72 amino acid residues of Bcr/Abl (green) comprises an N-terminal α-helix linked via a flexible loop to an elongated C-terminal α-helix that mediates tetramer formation. PMI in red is grafted to the short α-helical region in place of residues 5-16 of Bcr/Abl, resulting in ^{PMI}Bcr/Abl. To facilitate membrane permeabilization, ^{PMI}Bcr/Abl is C-terminally extended by an Arg-repeating hexapeptide (R6) in blue, yielding ^{PMI}Bcr/Abl-R6. ^{PMI}Bcr/Abl-R6 forms a stable tetramer, circulates in the blood, can accumulate in a tumor, can traverse a cell membrane, and can activate p53 by antagonizing MDM2/MDMX, leading to inhibition of tumor growth in animals.

### Synthesis and Biochemical and Biophysical Characterization of ^{PMI}Bcr/Abl-R6

Structural studies indicate that the N-terminal α-helix of Bcr/Abl (residues 5-15) does not contribute to protein tetramerization, which is mediated predominantly by the elongated C-terminal α-helix (residues 28-67) [34] (see, FIG. 2A). Since PMI (TSFAEYWALLSP; SEQ ID NO: 1) [38, 39] and residues 5-16 of Bcr/Abl (VGFAEAWKAQFP; SEQ ID NO: 2) share some degrees of sequence identity and structural similarity (see, FIG. 2A), the latter was replaced with the former in the Bcr/Abl amino acid sequence. Further, the C-terminus of Bcr/Abl was extended by an Arg-repeating hexapeptide (R6) to enhance cellular uptake, ultimately yielding ^{PMI}Bcr/Abl-R6 (MVDPTSFAEYWALLSPDSEPPRMELRSVGDIEQELERAKASIRRLEQEVNQERFRMIYLQTLLAK EKKSYDRRRRRRR, SEQ ID NO: 3) (see, FIG. 2B). ^{PMI}Bcr/Abl-R6 of 78 amino acid residues was chemically synthesized via native chemical ligation [40, 41] of two peptide fragments as illustrated in FIG. 2B and FIGS. 9A-9C. Ala38 was mutated to Cys to enable the ligation reaction, which was reverted to Ala, after ligation, though desulfurization as described [42]. The final product was purified by reversed phase HPLC and its molecular mass ascertained by electrospray ionization mass spectrometry (see, FIG. 2C). The synthetic proteins were folded by dissolving the polypeptides at 1 mg/ml in 6 M GuHCl, followed by a six-fold dilution with, and dialysis against, PBS containing 0.5 mM TCEP, pH 7.4. As shown in FIG. 2D, both Bcr/Abl-R6 and ^{PMI}Bcr/Abl-R6 adopted similar α-helical conformations in solution as evidenced by their similar circular dichroism spectra showing double minima at 208 and 222 nm and a positive peak at 195 nm, consistent with the known structural features of Bcr/Abl [34]. As negative controls, Bcr/Abl-R6 (MVDPVGFAEAWKAQFPDSEPPRMELRSVGDIEQELERAKASIRRLEQEVNQERFRMIYLQTLLA KEKKSYDRRRRRRR, SEQ ID NO: 4) and ^{PMI}Bcr/Abl (MVDPTSFAEYWALLSPDSEPPRMELRSVGDIEQELERAKASIRRLEQEVNQERFRMIYLQTLLAK EKKSYDR, SEQ ID NO: 5) were also chemically synthesized essentially as described for ^{PMI}Bcr/Abl-R6 (FIG. 2B).

Bcr/Abl-R6 and ^{PMI}Bcr/Abl-R6 were also characterized using size exclusion chromatography (FIG. 3A and FIGS. 10A-10B), dynamic light scattering (FIG. 3B and FIGS. 10A-10B), fluorescence polarization (FIG. 3C), and small angle X-ray scattering (FIGS. 3D-3E). All data unambiguously demonstrated that the synthetic proteins existed in aqueous buffer as tetramers at concentrations above 100 nM (FIG. 3C). Of note, measurements of the zeta potential of both ^{PMI}Bcr/Abl and ^{PMI}Bcr/Abl-R6 confirmed that the Arg-repeating hexapeptide R6 substantially increased protein surface charges, as expected (FIG. 11). Importantly, ^{PMI}Bcr/Abl-R6 was bound to the p53-binding domain of MDM2 with an affinity of 32 nM as measured by isothermal titration calorimetry (ITC) (FIG. 3F). By contrast, Bcr/Abl-R6 showed no binding to MDM2 under identical conditions (FIGS. 12A-12B). PMI, with a binding affinity of 0.52 nM for MDM2 (FIG. 3F) (K_{D}=0.5 nM, determined by surface plasmon resonance [38]), was significantly more potent than ^{PMI}Bcr/Abl-R6.

Isothermal titration calorimetry (ITC) data analysis was performed to measure the binding affinity of PMI and ^{PMI}Bcr/Abl-R6 for MDM2. Assays were performed on a MicroCal ITC 200 at 25° C. Concentrations of PMI and MDM2 were 100 μM and 10 μM, respectively. For the binding of ^{PMI}Bcr/Abl-R6 to MDM2, the concentrations 80 μM and 8 μM, respectively. The results are provided in Table 1, where it can be seen that despite a small net gain in entropy for ^{PMI}Bcr/Abl-R6 as opposed to a large loss for PMI, an expected outcome instigated by molecular grafting, ^{PMI}Bcr/Abl-R6 lost a substantial amount of enthalpy for binding, suggesting that structurally rigidified PMI in the context of Bcr/Abl was energetically suboptimal for MDM2 binding. Nevertheless, ITC-based binding assays clearly validated the molecular design at the functional level of a protein antagonist of MDM2.

TABLE 1
Measurements of the binding affinity of PMI and
^{PMI}Bcr/Abl-R6 for MDM2 by isothermal titration calorimetry (ITC).

| | PMI for MDM2 | ^{PMI}Bcr/Abl-R6 for MDM2 |
|---|---|---|
| N | 0.960 ± 0.004 | 0.987 ± 0.010 |
| ΔS | −29.4 cal/mol/deg | 2.33 cal/mol/deg |
| ΔH | −1.96E4 ± 344 cal/mol | −9207 ± 139 cal/mol |

^{PMI}Bcr/Abl-R6 with enhanced proteolytic stability efficiently permeabilizes HCT116 p53^{+/+} tumor cells via an endocytosis-independent pathway.

As was demonstrated previously [39], PMI was inactive in killing HCT116 p53^{+/+} cells due to its poor proteolytic stability and inability to traverse the cell membrane. Accordingly, the proteolytic stability of free PMI and ^{PMI}Bcr/Abl-R6 was compared in the presence of human serum (mainly serine proteases) or the intracellular cysteine protease cathepsin B. Intact peptide or protein was identified by mass spectrometry and quantified by RP-HPLC. As shown in FIGS. 4A-4B, the half-life of ^{PMI}Bcr/Abl-R6, compared with that of PMI, increased by 5-fold in human serum and 12-fold in cathepsin B. These data demonstrate that ^{PMI}Bcr/Abl-R6 is significantly more stable than free PMI in the presence of proteases.

Peptide/protein internalization and cytosolic release were also examined using both confocal microscopy and flow cytometry. As shown in FIG. 4C, flow cytometric analysis indicated that ^{PMI}Bcr/Abl-R6 N-terminally labeled with a BODIPY (borondipyrromethene) dye, BDP TR (589/616 nm), traversed HCT116 p53^{+/+} cell membranes much more efficiently than ^{PMI}Bcr/Abl or free PMI labeled with the same fluorophore. Confocal microscopic analysis confirmed this finding by showing the cytosolic distribution of ^{PMI}Bcr/Abl-R6, but not of ^{PMI}Bcr/Abl (FIG. 4D, panels A-C).

Cationic cell penetrating peptides as a carrier are known to promote cellular uptake of cargos primarily through the non-endocytic uptake pathway, or direct membrane translocation, which is inhibited by heparin but not by amiloride [43, 44]. To better understand the mechanism of cellular uptake of ^{PMI}Bcr/Abl-R6, confocal microscopic analysis was performed on cells treated with heparin or amiloride. As shown in FIG. 4D (panels D-E), while amiloride had little effect on ^{PMI}Bcr/Abl-R6 internalization, heparin almost completely blocked it, suggesting that the cellular uptake pathway for ^{PMI}Bcr/Abl-R6 is indeed endocytosis-independent.

^{PMI}Bcr/Abl-R6 Kills HCT116 p53^{+/+} Tumor Cells In Vitro by Reactivating the p53 Pathway.

To evaluate the tumor-killing activity of ^{PMI}Bcr/Abl-R6 in vitro, isogenic HCT116 p53^{+/+} and HCT116 p53^{-/-} cell lines expressing abundant MDM2 [45, 46] were treated with the ^{PMI}Bcr/Abl-R6 protein at concentrations from 1.56 μM to 50 μM. PMI and Bcr/Abl-R6 were used as a negative control and Nutlin-3, an extensively studied small molecule antagonist of MDM2 [47], as a positive control. As expected, while neither PMI nor Bcr/Abl-R6 had any effect on the viability of HCT116 cells 48 h after treatment (FIG. 5A and FIG. 13), a dose-dependent growth inhibition of HCT116 p53^{+/+} cells was observed with ^{PMI}Bcr/Abl-R6 and Nutlin-3 that were similarly active (FIG. 5A). Unlike ^{PMI}Bcr/Abl-R6, Nutlin-3 was toxic at 50 μM against HCT116 p53^{-/-} cells, indicative of its smaller therapeutic window than ^{PMI}Bcr/Abl-R6.

To investigate into the mechanisms of action of ^{PMI}Bcr/Abl-R6, the expression of p53, p21, PUMA and NOXA in HCT116 p53^{+/+} cells 48 h after treatment was analyzed by Western blotting. As shown in FIGS. 5B-5C, compared with mock-treated and PMI-, ^{PMI}Bcr/Abl- or Bcr/Abl-R6-treated cells, ^{PMI}Bcr/Abl-R6 or Nutlin-3 treatment significantly stabilized p53 in HCT116 p53^{+/+} cells, leading to upregulation of the p53-responsive genes p21, PUMA and NOXA important for cell cycle arrest and apoptosis [48, 49]. Consistent with these results, FACS analysis confirmed that HCT116 p53^{+/+} cells underwent similar degrees of apoptosis when treated with ^{PMI}Bcr/Abl-R6 or Nutlin-3, whereas mock-treated or Bcr/Abl-R6-treated cells were largely unaffected (FIGS. 5D-5E). Without being bound by theory, these data strongly suggest that ^{PMI}Bcr/Abl-R6 induces apoptosis of wild type p53-harboring tumor cells by antagonizing MDM2 to activate the p53 signaling pathway.

### ^{PMI}Bcr/Abl-R6 Accumulates and is Retained for an Extended Time in Solid Tumors In Vivo.

Nanoparticles can actively accumulate in solid tumors through leaky blood vessels in diseased tissues—a phenomenon known as the enhanced permeability and retention (EPR) effect [50, 51]. To examine the biodistribution of ^{PMI}Bcr/Abl-R6, the protein was fluorescently labeled with BDP TR and subcutaneously injected into BALB/c nude mice with palpable tumors grown from subcutaneously inoculated HCT116 p53^{+/+} cells. The biodistribution of ^{PMI}Bcr/Abl-R6 in the heart, lung, spleen, kidney, liver and tumor at three different time points (12, 24, and 48 h) was semi-quantitatively evaluated on an in vivo optical imaging system. As shown in FIGS. 6A-6B, the protein reached a maximum level at 24 h and accumulated predominantly in the kidney, liver and tumor. However, only the liver and tumor were found to harbor significant amounts of ^{PMI}Bcr/Abl-R6 at 48 h. Without being bound by theory, these data strongly suggest that ^{PMI}Bcr/Abl-R6 is capable of accumulating and is retained for an extended time in solid tumors, likely via the EPR effect.

^{PMI}Bcr/Abl-R6 Potently Inhibits Tumor Growth in Xenograft Mice by Inducing p53-Dependent Apoptotic Responses In Vivo.

To evaluate the therapeutic efficacy of ^{PMI}Bcr/Abl-R6 in vivo, a nude mouse xenograft model was established where animals were subcutaneously inoculated with HCT116 p53^{+/+} cells (3×10⁶). Thirty-six tumor-bearing mice were randomly divided into 6 groups (n=6) and received a 3-week subcutaneous treatment with medium, Nutlin-3, free PMI, Bcr/Abl-R6, ^{PMI}Bcr/Abl and ^{PMI}Bcr/Abl-R6 at the same dose of 5 mg/Kg every other day. As shown in FIGS. 7A-7D, while free PMI and Bcr/Abl-R6 had no effect on tumor growth, both Nutlin-3 and ^{PMI}Bcr/Abl-R6 significantly inhibited it. Interestingly, ^{PMI}Bcr/Abl was marginally active, suggesting that this protein, even without R6, was probably still able to partially traverse the cell membrane at high concentrations, albeit at a greatly reduced efficiency (FIG. 4C). It is important to point out that the molecular mass of ^{PMI}Bcr/Abl-R6 is 16-fold higher than that of Nutlin-3. The fact that ^{PMI}Bcr/Abl-R6 was even more effective than Nutlin-3 in inhibiting tumor growth suggests that ^{PMI}Bcr/Abl-R6, on the basis of molar concentration, is at least 16-fold more active as a monomer and 64-fold more active as a tetramer than Nutlin-3 in vivo.

Consistent with the above findings from the in vivo efficacy study, histopathological analysis using hematoxylin and eosin (H&E) (FIG. 7E) and terminal deoxynucleotidyl transferase dUTP nick end labeling (TUNEL) (FIG. 7F) staining techniques revealed massive necrotic and apoptotic tumor cells in the tissues from the ^{PMI}Bcr/Abl-R6-treated group and, to a lesser extent, in the tissues from the Nutlin-3-treated group. H&E and TUNEL staining also confirmed the partial activity of ^{PMI}Bcr/Abl and the lack of activity of PMI and Bcr/Abl-R6, as expected. Immunohistochemistry analysis demonstrated that ^{PMI}Bcr/Abl-R6 treatment significantly increased the expression of p53 and p21 in tumor tissues but decreased the expression of the tumor progression marker Ki-67 (FIG. 7G-7H). Taken together, the in vivo data unequivocally validates the design of ^{PMI}Bcr/Abl-R6 as a potent antitumor agent that inhibits tumor growth in a p53-dependent fashion.

### ^{PMI}Bcr/Abl-R6 is Minimally Immunogenic and Non-Toxic to Blood Cells and Kidney and Liver Tissues.

Immunogenicity of peptide/protein therapeutics often impedes their clinical use. The immunogenicity of PMI and ^{PMI}Bcr/Abl-R6 in immune-competent C57BL/6 mice was evaluated by measuring the level of the cytokines IL-2, TNF-α and erythropoietin (EPO) in the blood in response to subcutaneous treatments with PMI and ^{PMI}Bcr/Abl-R6 for three weeks, every other day, at a dose of 5 mg/Kg. IL-2 and TNF-α were used as markers because T cell responses are known to play a critical role in the development of immunogenic responses to therapeutic peptides and proteins [52, 53]. Since biotherapeutics can potentially generate cross-reactive neutralizing antibodies that inhibit endogenous proteins such as EPO, leading to anemia known as antibody-mediated pure red-cell aplasia, EPO was also used as a marker for immunogenicity in the study. As shown in FIGS. 8A-8C, while free PMI noticeably increased IL-2 and TNF-α levels and decreased the level of EPO, only slight changes in the amount of IL-2, TNF-α and EPO were observed with ^{PMI}Bcr/Abl-R6, suggesting that grafting PMI to the Bcr/Abl protein scaffold significantly dampened immunogenicity of the peptide drug.

Some small molecule antagonists of MDM2 have showed cytotoxicity against B lymphocytes and hematopoietic stem cells in clinical trials, resulting in side effects such as thrombocytopenia, leukopenia and neutropenia [54]. The cytotoxicity profile of PMI, Bcr/Abl-R6, ^{PMI}Bcr/Abl, ^{PMI}Bcr/Abl-R6 and Nutlin-3 was established at the end of the three-week treatment by counting white blood cells, lymphocytes, monocytes, granulocytes, red blood cells, and platelets in a complete blood cell analysis. As shown in FIG. 8D, no statistically significant difference in the number of each cell type was observed for all five treatment groups compared with the mock-treated control group.

Since ^{PMI}Bcr/Abl-R6 accumulates in the liver and kidney in addition to the solid tumor (FIG. 6), the two principal organs for drug metabolism and elimination, the in vivo toxicity of PMI, Bcr/Abl-R6, ^{PMI}Bcr/Abl, ^{PMI}Bcr/Abl-R6 and Nutlin-3 to liver and kidney tissues was also examined by H&E staining at the end of the three-week treatment. As shown in FIG. 8E, no overt toxicity was observed at the doses used in the study. Taken together, the in vivo immunogenicity and toxicity data validate the safety of ^{PMI}Bcr/Abl-R6.

### REFERENCES

[1] T. Ideker, R. Sharan, Protein networks in disease, Genome research 18(4) (2008) 644-652.
[2] U. Stelzl, U. Worm, M. Lalowski, C. Haenig, F. H. Brembeck, H. Goehler, M. Stroedicke, M. Zenkner, A. Schoenherr, S. Koeppen, A human protein-protein interaction network: a resource for annotating the proteome, Cell 122(6) (2005) 957-968.
[3] L.-G. Milroy, T. N. Grossmann, S. Hennig, L. Brunsveld, C. Ottmann, Modulators of protein-protein interactions, Chemical reviews 114(9) (2014) 4695-4748.
[4] A.-L. Barabási, N. Gulbahce, J. Loscalzo, Network medicine: a network-based approach to human disease, Nature reviews genetics 12(1) (2011) 56.
[5] D. J. Craik, D. P. Fairlie, S. Liras, D. Price, The future of peptide-based drugs, Chemical biology & drug design 81(1) (2013) 136-147.
[6] D. E. Scott, A. R. Bayly, C. Abell, J. Skidmore, Small molecules, big targets: drug discovery faces the protein-protein interaction challenge, Nature Reviews Drug Discovery 15(8) (2016) 533.
[7] P. Vlieghe, V. Lisowski, J. Martinez, M. Khrestchatisky, Synthetic therapeutic peptides: science and market, Drug discovery today 15(1-2) (2010) 40-56.
[8] L. D. Walensky, A. L. Kung, I. Escher, T. J. Malia, S. Barbuto, R. D. Wright, G. Wagner, G. L. Verdine, S. J. Korsmeyer, Activation of apoptosis in vivo by a hydrocarbon-stapled BH3 helix, Science 305(5689) (2004) 1466-1470.
[9] J. A. Robinson, β-Hairpin peptidomimetics: design, structures and biological activities, Accounts of chemical research 41(10) (2008) 1278-1288.
[10] R. N. Zuckermann, T. Kodadek, Peptoids as potential therapeutics, Curr. Opin. Mol. Ther 11(3) (2009) 299-307.
[11] R. J. Simon, R. S. Kania, R. N. Zuckermann, V. D. Huebner, D. A. Jewell, S. Banville, S. Ng, L. Wang, S. Rosenberg, C. K. Marlowe, Peptoids: a modular approach to drug discovery, Proceedings of the National Academy of Sciences 89(20) (1992) 9367-9371.
[12] N. L. Daly, D. J. Craik, Bioactive cystine knot proteins, Current opinion in chemical biology 15(3) (2011) 362-368.
[13] K. Gradauer, J. Barthelmes, C. Vonach, G. Almer, H. Mangge, B. Teubl, E. Roblegg, S. Dünnhaupt, E. Fröhlich, A. Bernkop-Schnürch, Liposomes coated with thiolated chitosan enhance oral peptide delivery to rats, Journal of controlled release 172(3) (2013) 872-878.
[14] Z. Niu, I. Conejos-Sánchez, B. T. Griffin, C. M. O'Driscoll, M. J. Alonso, Lipid-based nanocarriers for oral peptide delivery, Advanced drug delivery reviews 106 (2016) 337-354.
[15] M. Liu, M. Pazgier, C. Li, W. Yuan, C. Li, W. Lu, A Left-Handed Solution to Peptide Inhibition of the p53-MDM2 Interaction, Angewandte Chemie International Edition 49(21) (2010) 3649-3652.
[16] Y. Shi, P. Teng, P. Sang, F. She, L. Wei, J. Cai, gamma-AApeptides: Design, Structure, and Applications, Acc Chem Res 49(3) (2016) 428-41.
[17] S. Mitragotri, P. A. Burke, R. Langer, Overcoming the challenges in administering biopharmaceuticals: formulation and delivery strategies, Nature reviews Drug discovery 13(9) (2014) 655.
[18] M. L. Tan, P. F. Choong, C. R. Dass, Recent developments in liposomes, microparticles and nanoparticles for protein and peptide drug delivery, Peptides 31(1) (2010) 184-193.
[19] S. Mitra, J. E. Montgomery, M. J. Kolar, G. Li, K. J. Jeong, B. Peng, G. L. Verdine, G. B. Mills, R. E. Moellering, Stapled peptide inhibitors of RAB25 target context-specific phenotypes in cancer, Nature communications 8(1) (2017) 660.
[20] F. Bernal, M. Wade, M. Godes, T. N. Davis, D. G. Whitehead, A. L. Kung, G. M. Wahl, L. D. Walensky, A stapled p53 helix overcomes HDMX-mediated suppression of p53, Cancer cell 18(5) (2010) 411-422.
[21] M. Liu, C. Li, M. Pazgier, C. Li, Y. Mao, Y. Lv, B. Gu, G. Wei, W. Yuan, C. Zhan, D-peptide inhibitors of the p53-MDM2 interaction for targeted molecular therapy of malignant neoplasms, Proceedings of the National Academy of Sciences 107(32) (2010) 14321-14326.
[22] J. Shi, A. R. Votruba, O. C. Farokhzad, R. Langer, Nanotechnology in drug delivery and tissue engineering: from discovery to applications, Nano letters 10(9) (2010) 3223-3230.
[23] C. Wang, L. Cheng, Z. Liu, Drug delivery with upconversion nanoparticles for multi-functional targeted cancer cell imaging and therapy, Biomaterials 32(4) (2011) 1110-1120.
[24] Y. Ma, X. Liang, S. Tong, G. Bao, Q. Ren, Z. Dai, Gold Nanoshell Nanomicelles for Potential Magnetic Resonance Imaging, Light-Triggered Drug Release, and Photothermal Therapy, Advanced functional materials 23(7) (2013) 815-822.
[25] W. T. Al-Jamal, K. Kostarelos, Liposomes: from a clinically established drug delivery system to a nanoparticle platform for theranostic nanomedicine, Accounts of chemical research 44(10) (2011) 1094-1104.
[26] A. Altunbas, S. J. Lee, S. A. Rajasekaran, J. P. Schneider, D. J. Pochan, Encapsulation of curcumin in self-assembling peptide hydrogels as injectable drug delivery vehicles, Biomaterials 32(25) (2011) 5906-5914.
[27] A. O. Elzoghby, W. M. Samy, N. A. Elgindy, Protein-based nanocarriers as promising drug and gene delivery systems, Journal of controlled release 161(1) (2012) 38-49.
[28] W. Lohcharoenkal, L. Wang, Y. C. Chen, Y. Rojanasakul, Protein nanoparticles as drug delivery carriers for cancer therapy, BioMed research international 2014 (2014).
[29] A. O. Elzoghby, W. M. Samy, N. A. Elgindy, Albumin-based nanoparticles as potential controlled release drug delivery systems, Journal of controlled release 157(2) (2012) 168-182.
[30] Q. Peng, S. Zhang, Q. Yang, T. Zhang, X.-Q. Wei, L. Jiang, C.-L. Zhang, Q.-M. Chen, Z.-R. Zhang, Y.-F. Lin, Preformed albumin corona, a protective coating for nanoparticles based drug delivery system, Biomaterials 34(33) (2013) 8521-8530.
[31] L. Y. Chan, S. Gunasekera, S. T. Henriques, N. F. Worth, S.-J. Le, R. J. Clark, J. H. Campbell, D. J. Craik, N. L. Daly, Engineering pro-angiogenic peptides using stable, disulfide-rich cyclic scaffolds, Blood 118(25) (2011) 6709-6717.
[32] C. Li, M. Pazgier, M. Liu, W. Y. Lu, W. Lu, Apamin as a Template for Structure-Based Rational Design of Potent Peptide Activators of p53, Angewandte Chemie International Edition 48(46) (2009) 8712-8715.
[33] C. Li, M. Liu, J. Monbo, G. Zou, C. Li, W. Yuan, D. Zella, W.-Y. Lu, W. Lu, Turning a scorpion toxin into an antitumor miniprotein, Journal of the American Chemical Society 130(41) (2008) 13546-13548.
[34] X. Zhao, S. Ghaffari, H. Lodish, V. N. Malashkevich, P. S. Kim, Structure of the Bcr-Abl oncoprotein oligomerization domain, Nature Structural and Molecular Biology 9(2) (2002) 117.
[35] K. H. Khoo, C. S. Verma, D. P. Lane, Drugging the p53 pathway: understanding the route to clinical efficacy, Nature reviews Drug discovery 13(3) (2014) 217-236.
[36] Q. Zhang, S. X. Zeng, H. Lu, Targeting p53-MDM2-MDMX loop for cancer therapy, Mutant p53 and MDM2 in Cancer, Springer 2014, pp. 281-319.
[37] M. Wade, Y. C. Li, G. M. Wahl, MDM2, MDMX and p53 in oncogenesis and cancer therapy, Nat Rev Cancer 13(2) (2013) 83-96.
[38] C. Li, M. Pazgier, C. Li, W. Yuan, M. Liu, G. Wei, W.-Y. Lu, W. Lu, Systematic mutational analysis of peptide inhibition of the p53-MDM2/MDMX interactions, Journal of molecular biology 398(2) (2010) 200-213.
[39] M. Pazgier, M. Liu, G. Zou, W. Yuan, C. Li, C. Li, J. Li, J. Monbo, D. Zella, S. G. Tarasov, Structural basis for high-affinity peptide inhibition of p53 interactions with MDM2 and MDMX, Proceedings of the National Academy of Sciences 106(12) (2009) 4665-4670.
[40] P. E. Dawson, T. W. Muir, I. Clark-Lewis, S. Kent, Synthesis of proteins by native chemical ligation, Science 266(5186) (1994) 776-779.
[41] P. E. Dawson, S. B. Kent, Synthesis of native proteins by chemical ligation, Annual review of biochemistry 69(1) (2000) 923-960.
[42] L. Z. Yan, P. E. Dawson, Synthesis of peptides and proteins without cysteine residues by native chemical ligation combined with desulfurization, Journal of the American Chemical Society 123(4) (2001) 526-533.
[43] L. N. Patel, J. L. Zaro, W.-C. Shen, Cell penetrating peptides: intracellular pathways and pharmaceutical perspectives, Pharmaceutical research 24(11) (2007) 1977-1992.
[44] H. L. Åmand, H. A. Rydberg, L. H. Fornander, P. Lincoln, B. Nordén, E. K. Esbjörner, Cell surface binding and uptake of arginine-and lysine-rich penetratin peptides in absence and presence of proteoglycans, Biochimica et Biophysica Acta (BBA)-Biomembranes 1818(11) (2012) 2669-2678.
[45] R. K. Geyer, K. Y. Zhong, C. G. Maki, The MDM2 RING-finger domain is required to promote p53 nuclear export, Nature cell biology 2(9) (2000) 569.
[46] S. Sur, R. Pagliarini, F. Bunz, C. Rago, L. A. Diaz, K. W. Kinzler, B. Vogelstein, N. Papadopoulos, A panel of isogenic human cancer cells suggests a therapeutic approach for cancers with inactivated p53, Proceedings of the National Academy of Sciences 106(10) (2009) 3964-3969.
[47] L. T. Vassilev, B. T. Vu, B. Graves, D. Carvajal, F. Podlaski, Z. Filipovic, N. Kong, U. Kammlott, C. Lukacs, C. Klein, In vivo activation of the p53 pathway by small-molecule antagonists of MDM2, Science 303(5659) (2004) 844-848.
[48] Y. Xiong, G. J. Hannon, H. Zhang, D. Casso, R. Kobayashi, D. Beach, p21 is a universal inhibitor of cyclin kinases, nature 366(6456) (1993) 701.
[49] A. Villunger, E. M. Michalak, L. Coultas, F. Miillauer, G. Böck, M. J. Ausserlechner, J. M. Adams, A. Strasser, p53-and drug-induced apoptotic responses mediated by BH3-only proteins puma and noxa, Science 302(5647) (2003) 1036-1038.
[50] H. Maeda, H. Nakamura, J. Fang, The EPR effect for macromolecular drug delivery to solid tumors: Improvement of tumor uptake, lowering of systemic toxicity, and distinct tumor imaging in vivo, Advanced drug delivery reviews 65(1) (2013) 71-79.
[51] V. Torchilin, Tumor delivery of macromolecular drugs based on the EPR effect, Advanced drug delivery reviews 63(3) (2011) 131-135.
[52] V. Jawa, L. P. Cousens, M. Awwad, E. Wakshull, H. Kropshofer, A. S. De Groot, T-cell dependent immunogenicity of protein therapeutics: preclinical assessment and mitigation, Clinical immunology 149(3) (2013) 534-555.
[53] A. S. De Groot, D. W. Scott, Immunogenicity of protein therapeutics, Trends in immunology 28(11) (2007) 482-490.
[54] A. Burgess, K. M. Chia, S. Haupt, D. Thomas, Y. Haupt, E. Lim, Clinical Overview of MDM2/X-Targeted Therapies, Front Oncol 6 (2016) 7.
[55] K. H. Khoo, C. S. Verma, D. P. Lane, Drugging the p53 pathway: understanding the route to clinical efficacy, Nature reviews. Drug discovery 13(3) (2014) 217-36.
[56] Q. Zhang, S. X. Zeng, H. Lu, Targeting p53-MDM2-MDMX loop for cancer therapy, Subcell Biochem 85 (2014) 281-319.
[57] N. Estrada-Ortiz, C. G. Neochoritis, A. Domling, How To Design a Successful p53-MDM2/X Interaction Inhibitor: A Thorough Overview Based on Crystal Structures, ChemMedChem 11(8) (2016) 757-72.
[58]Y. Zhao, A. Aguilar, D. Bernard, S. Wang, Small-molecule inhibitors of the MDM2-p53 protein-protein interaction (MDM2 Inhibitors) in clinical trials for cancer treatment, J Med Chem 58(3) (2015) 1038-52.
[59] F. Meric-Bernstam, M. N. Saleh, J. R. Infante, S. Goel, G. S. Falchook, G. Shapiro, K. Y. Chung, R. M. Conry, D. S. Hong, J. S.-Z. Wang, U. Steidl, L. D. Walensky, V. Guerlavais, M. Payton, D. A. Annis, M. Aivado, M. R. Patel, Phase I trial of a novel stapled peptide ALRN-6924 disrupting MDMX- and MDM2-mediated inhibition of WT p53 in patients with solid tumors and lymphomas, Journal of Clinical Oncology 35(15_suppl) (2017) 2505-2505.
[60] L. A. Carvajal, D. B. Neriah, A. Senecal, L. Benard, V. Thiruthuvanathan, T. Yatsenko, S. R. Narayanagari, J. C. Wheat, T. I. Todorova, K. Mitchell, C. Kenworthy, V. Guerlavais, D. A. Annis, B. Bartholdy, B. Will, J. D. Anampa, I. Mantzaris, M. Aivado, R. H. Singer, R. A. Coleman, A. Verma, U. Steidl, Dual inhibition of MDMX and MDM2 as a therapeutic strategy in leukemia, Sci Transl Med 10(436) (2018).
[61] F. Bernal, A. F. Tyler, S. J. Korsmeyer, L. D. Walensky, G. L. Verdine, Reactivation of the p53 tumor suppressor pathway by a stapled p53 peptide, J Am Chem Soc 129(9) (2007) 2456-7.
[62] G. L. Verdine, G. J. Hilinski, Stapled peptides for intracellular drug targets, Methods Enzymol 503 (2012) 3-33.
[63] C. J. Brown, S. T. Quah, J. Jong, A. M. Goh, P. C. Chiam, K. H. Khoo, M. L. Choong, M. A. Lee, L. Yurlova, K. Zolghadr, T. L. Joseph, C. S. Verma, D. P. Lane, Stapled peptides with improved potency and specificity that activate p53, ACS Chem Biol 8(3) (2013) 506-12.
[64] X. Chen, L. Tai, J. Gao, J. Qian, M. Zhang, B. Li, C. Xie, L. Lu, W. Lu, W. Lu, A stapled peptide antagonist of MDM2 carried by polymeric micelles sensitizes glioblastoma to temozolomide treatment through p53 activation, J Control Release 218 (2015) 29-35.
[65] X. Li, W. D. Tolbert, H.-G. Hu, N. Gohain, Y. Zou, F. Niu, W.-X. He, W. Yuan, J.-C. Su, M. Pazgier, W. Lu, Dithiocarbamate-inspired side chain stapling chemistry for peptide drug design Chemical science (2019).
[66] M. Schnolzer, P. Alewood, A. Jones, D. Alewood, S. B. Kent, In situ neutralization in Boc-chemistry solid phase peptide synthesis. Rapid, high yield assembly of difficult sequences, Int J Pept Protein Res 40(3-4) (1992) 180-93.
[67] C. N. Pace, F. Vajdos, L. Fee, G. Grimsley, T. Gray, How to measure and predict the molar absorption coefficient of a protein, Protein Sci 4(11) (1995) 2411-23.
[68] Y.-H. Chen, J. T. Yang, H. M. Martinez, Determination of the secondary structures of proteins by circular dichroism and optical rotatory dispersion, Biochemistry 11(22) (1972) 4120-4131.

1. A protein comprising a protein scaffold and at least one therapeutic peptide grafted therein, wherein the protein scaffold is a disulfide-devoid tetramerization domain of chimeric oncoprotein Bcr/Abl protein of chronic myeloid leukemia, as defined in SEQ ID NO: 6.

2. The protein of claim 1, wherein the therapeutic peptide has an α-helical structure.

3. The protein of claim 1, wherein the therapeutic peptide is grafted into the N-terminus of the Bcr/Abl protein.

4. The protein of claim 1, wherein the therapeutic peptide antagonizes intracellular MDM2/MDMX, thereby activating p53.

5. The protein of claim 1, wherein the therapeutic peptide is PMI grafted in place of residues 5-16 of the Bcr/Abl protein.

6. The protein of claim 1, further comprising a C-terminal extension to allow the protein to traverse a cell membrane.

7. The protein of claim 6, wherein the C-terminal extension is an Arg-repeating hexapeptide (R6).

8. A ^{PMI}Bcr/Abl protein comprising a sequence as shown in SEQ ID NO: 5.

9. The ^{PMI}Bcr/Abl protein of claim 8, further comprising a C-terminal extension to allow the protein to traverse a cell membrane.

10. The ^{PMI}Bcr/Abl protein of claim 9, wherein the C-terminal extension is an Arg-repeating hexapeptide (R6).

11. A ^{PMI}Bcr/Abl-R6 protein comprising a sequence as shown in SEQ ID NO: 3.

12. A method of inhibiting tumor cell growth in a mammal, said method comprising administering a protein of claim 1 to said mammal.

13. The method of claim 12, wherein the protein antagonizes intracellular MDM2/MDMX, thereby activating p53.

14. A method of inducing apoptosis of cancer cells in a mammal, said method comprising administering a protein of claim 1 to said mammal.

15. The method of claim 14, wherein the protein antagonizes intracellular MDM2/MDMX, thereby activating p53.

16. A method of treating Philadelphia chromosome-positive acute lymphocytic leukemia (ALL) and/or chronic myelogenous leukemia (CML) in a mammal, said method comprising administering a protein of claim 1 to a mammal.

17. The method of claim 16, wherein the ALL and/or CML are resistant to imatinib.

18. A method of delivering a p53-activating compound for cancer treatment, said method comprising administering a protein of claim 1 to a mammal.
